SEQUENCE LISTING (14 sequences)

SEQ ID NO: 1 - protein, 195 aa, synthetic construct
  PEPTIDE 1..195: note = recombinant oncostatin M protein
AAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSR

SEQ ID NO: 2 - protein, 183 aa, synthetic construct
  PEPTIDE 1..183: note = recombinant IL-6 protein
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - DNA, 20 bp, synthetic construct
  source 1..20: mol_type = other DNA
  misc_feature 1..20: note = Forward (5 prime-3 prime) Oligonucleotide
acaacctggagaacttcaac

SEQ ID NO: 4 - DNA, 21 bp, synthetic construct
  source 1..21: mol_type = other DNA
  misc_feature 1..21: note = Forward (5 prime-3 prime) Oligonucleotide
tgcccggctacatgaataaca

SEQ ID NO: 5 - DNA, 18 bp, synthetic construct
  source 1..18: mol_type = other DNA
  misc_feature 1..18: note = Forward (5 prime-3 prime) Oligonucleotide
acgcagcagcactactac

SEQ ID NO: 6 - DNA, 20 bp, synthetic construct
  source 1..20: mol_type = other DNA
  misc_feature 1..20: note = Forward (5 prime-3 prime) Oligonucleotide
tggcatcaacgctgtcttct

SEQ ID NO: 7 - DNA, 22 bp, synthetic construct
  source 1..22: mol_type = other DNA
  misc_feature 1..22: note = Forward (5 prime-3 prime) Oligonucleotide
ctgaggacgtggagaaaacttg

SEQ ID NO: 8 - DNA, 21 bp, synthetic construct
  source 1..21: mol_type = other DNA
  misc_feature 1..21: note = Forward (5 prime-3 prime) Oligonucleotide
cagtggcttggaaggaaaagg

SEQ ID NO: 9 - DNA, 20 bp, synthetic construct
  source 1..20: mol_type = other DNA
  misc_feature 1..20: note = Reverse (5 prime-3 prime) Oligonucleotide
atctgggacacagtagtgaa

SEQ ID NO: 10 - DNA, 21 bp, synthetic construct
  source 1..21: mol_type = other DNA
  misc_feature 1..21: note = Reverse (5 prime-3 prime] Oligonucleotide
tgtcattgggatagaggaccc

SEQ ID NO: 11 - DNA, 18 bp, synthetic construct
  source 1..18: mol_type = other DNA
  misc_feature 1..18: note = Reverse (5 prime-3 prime) Oligonucleotide
tcgcaggcattctgaagg

SEQ ID NO: 12 - DNA, 20 bp, synthetic construct
  source 1..20: mol_type = other DNA
  misc_feature 1..20: note = Reverse (5 prime-3 prime) Oligonucleotide
agccaatggtggcatacaca

SEQ ID NO: 13 - DNA, 24 bp, synthetic construct
  source 1..24: mol_type = other DNA
  misc_feature 1..24: note = Reverse (5 prime-3 prime) Oligonucleotide
aatatcagagctggggtgaccttc

SEQ ID NO: 14 - DNA, 20 bp, synthetic construct
  source 1..20: mol_type = other DNA
  misc_feature 1..20: note = Reverse (5 prime-3 prime) Oligonucleotide
caggtgagtgggagcaatct

(19) United States
(12) Patent Application Publication
(10) Pub. No.: US 2026/0199289 A1
(43) Pub. Date: Jul. 16, 2026

(54) USE OF GLYCOGEN METABOLISM INHIBITORS FOR THE TREATMENT OF CANCER

(71) Applicant: THE UNIVERSITY OF VERMONT AND STATE AGRICULTURAL COLLEGE, Burlington, VT (US)

(72) Inventors: Frances E. Carr, Shelburne, VT (US); Cole D. Davidson, South Burlington, VT (US)

(21) Appl. No.: 19/099,742

(22) PCT Filed: Jul. 28, 2023
(86) PCT No.: PCT/US2023/071213

§ 371 Date: Jan. 29, 2025

Related U.S. Application Data
(60) Provisional application No. 63/393,546, filed on Jul. 29, 2022.

(51) Int. Cl. A61K 31/404; A61K 31/17; A61K 31/225; A61K 31/4355; A61K 31/4439; A61K 31/454; A61K 31/47; A61K 31/496; A61K 31/506; A61K 31/7008; A61P 35/00; C07K 16/22

(52) U.S. Cl. CPC A61K 31/404; A61K 31/17; A61K 31/225; A61K 31/4355; A61K 31/4439; A61K 31/454; A61K 31/47; A61K 31/496; A61K 31/506; A61K 31/7008; A61P 35/00; C07K 16/22

(57) ABSTRACT

Disclosed herein is a method of treating cancer in a subject that includes administering to a subject having a cancer an inhibitor of glycogen metabolism under conditions effective to treat the cancer, wherein the cancer is characterized by cells having an increased glycogen expression or activity relative to corresponding non-cancer cells of similar origin. Further disclosed herein is a method of inhibiting tumor growth in a subject that includes administering to a subject having a tumor an inhibitor of glycogen metabolism under conditions effective to treat the tumor, wherein the tumor is characterized by cells having an increased glycogen expression or activity relative to corresponding non-tumor cells of similar origin. The present disclosure further relates to a combination therapy that includes an inhibitor of glycogen metabolism and a cancer therapeutic.

This application claims the benefit of U.S. Provisional patent application Ser. No. 63/393,546, filed Jul. 29, 2022, which is hereby incorporated by reference in its entirety.

### GOVERNMENT FUNDING

This invention was made with Government support under Grant Number U54 GM115516 awarded by the National Institutes for Health. The United States Government has certain rights in the invention.

### FIELD

The present disclosure is directed to methods and compositions comprising glycogen metabolism inhibitors for use in the treatment of cancer.

### SEQUENCE LISTING

The instant application contains a computer readable Sequence Listing which has been submitted electronically in XML format (“Sequence Listing XML”) and is hereby incorporated by reference in its entirety. The Sequence Listing, created on Jul. 27, 2023, is named 147405.000171.xml and is 19,328 bytes in size.

### BACKGROUND

Thyroid cancer is the most common malignancy of the endocrine system, and the incidence has greatly increased over the past forty years. Reem ElSheikh, S. M., “The Incidence of Clinically Relevant Thyroid Cancers Remains Stable,” Clinical Thyroidology 34:26-28 (2022). Well-differentiated thyroid cancers such as papillary (PTC) and low-grade follicular (FTC) thyroid cancers can usually be treated with surgery, radiation, or radioactive iodide (RAI) therapy. However, poorly differentiated (PDTC) and anaplastic thyroid cancers (ATC) are highly aggressive, metastatic, stem-like, and develop resistance to commonly used therapies. Lin et al., “The Incidence and Survival Analysis for Anaplastic Thyroid Cancer: A Seer Database Analysis,” Am J Transl Res 11:5888-5896 (2019). Sorafenib is commonly used in ATC patients to target BRAFV600E, a gain of function mutation found in nearly 50% of ATC tumors. Yoo et al., “Integrative Analysis of Genomic and Transcriptomic Characteristics Associated With Progression of Aggressive Thyroid Cancer,” Nature Communications 10:2764 (2019). Unfortunately, escape mechanisms arise that render sorafenib ineffective just months after application. Jayarangaiah et al., “Therapeutic Options for Advanced Thyroid Cancer,” Int J Clin Endocrinol Metab 5:26-34 (2019); Krajewska et al., “Sorafenib for the Treatment of Thyroid Cancer: An Updated Review,” Expert Opin Pharmacother 16:573-583 (2015); Naoum et al., “Novel Targeted Therapies and Immunotherapy for Advanced Thyroid Cancers,” Mol Cancer 17:51-51 (2018); and Valerio et al., “Targeted Therapy in Thyroid Cancer: State of the Art,” Clin Oncol (R Coll Radiol) 29:316-324 (2017). Therefore, there is an urgent need to address the abysmal clinical outcome for one of the most lethal tumors.

Historically, targeted therapies in poorly differentiated thyroid cancer and ATC have inhibited cell signaling kinases with varying levels of success. However, few strategies have been rigorously tested to target metabolic alterations in thyroid cancer. Davidson, C. D., and Carr, F. E., “Review of Pharmacological Inhibition of Thyroid Cancer Metabolism,” Journal of Cancer Metastasis and Treatment 7:45 (2021). Many cancer cells preferentially utilize glycolysis over oxidative phosphorylation even in the presence of oxygen, a phenomenon known as the Warburg effect. Potter et al., “The Warburg Effect: 80 Years on,” Biochemical Society Transactions 44:1499-1505 (2016). The high rate of glycolysis in cancer cells may be to generate lactate to acidify the tumor microenvironment, favoring angiogenesis and immunosuppression, while generating nucleotides and lipids for cell division. Hexokinase has been identified as an attractive drug target to limit the first step in glycolysis. The hexokinase inhibitor 2-deoxyglucose (2-DG) reduced lactate production in an ATC xenograft but failed to significantly reduce tumor size. Sandulache et al., “Glycolytic Inhibition Alters Anaplastic Thyroid Carcinoma Tumor Metabolism and Improves Response to Conventional Chemotherapy and Radiation.” Molecular Cancer Therapeutics 11:1373-1380 (2012). Additionally, 2-DG treatment levies concern for long-term clinical application due to its broad-spectrum toxicity, particularly in the brain, heart, and stomach. Laussel, C., and Léon, S., “Cellular Toxicity of the Metabolic Inhibitor 2-deoxyglucose and Associated Resistance Mechanisms,” Biochem Pharmacol 182:114213 (2020). The pan-metabolic inhibitor metformin has been shown to limit ATC proliferation in vitro and is in a phase II clinical trial for differentiated thyroid cancer in combination with RAI. Chen et al., “Synergistic Anti-proliferative Effect of Metformin and Sorafenib on Growth of Anaplastic Thyroid Cancer Cells and Their Stem Cells,” Oncology Reports 33:1994-2000 (2015); Chen et al., “Variations in Glycogen Synthesis in Human Pluripotent Stem Cells With Altered Pluripotent States,” PLoS One 10:e0142554-e0142554 (2015); and Davidson, C. D., and Carr, F. E., “Review of Pharmacological Inhibition of Thyroid Cancer Metabolism,” Journal of Cancer Metastasis and Treatment 7:45 (2021). However, metformin has many metabolic targets, and the therapeutic efficacy in cancer patients remains controversial. Akhter, M. S., and Uppal, P., “Toxicity of Metformin and Hypoglycemic Therapies,” Adv Chronic Kidney Dis 27:18-30 (2020) and Pernicova, I., and Korbonits, M., “Metformin—Mode of Action and Clinical Implications for Diabetes and Cancer,” Nature Reviews Endocrinology 10:143-156 (2014). Ideally, a metabolic inhibitor would exhibit low toxicity by inhibiting a metabolic pathway that is active only in a few cell types in normal tissue, in addition to the cancer cells being targeted.

In addition to importing glucose from the bloodstream, many tumors have been shown to fuel cell metabolism from storing and breaking down the glucose polymer, glycogen. Favaro et al., “Glucose Utilization Via Glycogen Phosphorylase Sustains Proliferation and Prevents Premature Senescence in Cancer Cells,” Cell Metabolism 16:751-764 (2012); Lea et al., “Glycogen Metabolism in Regenerating Liver and Liver Neoplasms,” Cancer Research 32:61-66 (1972); Markopoulos et al., “Glycogen-rich Clear Cell Carcinoma of the Breast,” World Journal of Surgical Oncology 6:44 (2008); and Rousset et al., “Presence of Glycogen and Growth-related Variations in 58 Cultured Human Tumor Cell Lines of Various Tissue Origins,” Cancer Research 41:1165-1170 (1981). Glucose can be stored as a quick-access carbon cache via glycogen synthase 1 (GYS1) and rapidly broken back down to glucose via glycogen phosphorylase liver (PYGL) or brain (PYGB) isoforms. Glycogen deposits have been clinically observed in diverse types of cancer and have been shown to play a role in tumor progression. Barot et al., “Inhibition of Glycogen Catabolism induces Intrinsic Apoptosis and Augments Multikinase Inhibitors in Hepatocellular Carcinoma Cells,” Experimental Cell Research 381(2):288-300 (2019); Dauer, P., and Lengyel, E., “New Roles for Glycogen in Tumor Progression,” Trends Cancer 5:396-399 (2019); Favaro et al., “Glucose Utilization Via Glycogen Phosphorylase Sustains Proliferation and Prevents Premature Senescence in Cancer Cells,” Cell Metabolism 16:751-764 (2012); and Lee et al., “Metabolic Sensitivity of Pancreatic Tumour Cell Apoptosis to Glycogen Phosphorylase Inhibitor Treatment,” British Journal of Cancer 91:2094-2100 (2004). Furthermore, inhibiting glycogen breakdown via RNAi has shown remarkable success in a glioblastoma xenograft model. Favaro et al., “Glucose Utilization Via Glycogen Phosphorylase Sustains Proliferation and Prevents Premature Senescence in Cancer Cells,” Cell Metabolism 16:751-764 (2012). While glycogen represents a promising metabolic target in cancer, glycogen stores have only been reported in normal canine and bovine thyroids and the very rare clear cell thyroid carcinoma. AHN, C. S., “Glycogen Metabolism of the Thyroid,” Endocrinology 88:1341-1348 (1971); Carcangiu et al., “Clear Cell Change in Primary Thyroid Tumors. A Study of 38 Cases.” Am J Surg Pathol 9:705-722 (1985); and Juhlin, C. C., and Höög, A., “Clear Cell Variant of Papillary Thyroid Carcinoma With Associated Anaplastic Thyroid Carcinoma: Description of an Extraordinary Case,” Int J Surg Pathol 27:658-663 (2019).

The present disclosure is directed at overcoming these deficiencies in the field by providing a combination therapy for the treatment of early stage, aggressive, and treatment-resistant forms of cancer.

### SUMMARY

A first aspect of the present disclosure relates to a method of treating cancer in a subject. The method includes administering to a subject having a cancer an inhibitor of glycogen metabolism under conditions effective to treat the cancer, wherein the cancer is characterized by cells having an increased glycogen expression or activity relative to corresponding non-cancer cells of similar origin.

Another aspect of the present disclosure is directed to a method of inhibiting tumor growth in a subject. The method includes administering to a subject having a tumor an inhibitor of glycogen metabolism under conditions effective to treat the tumor, wherein the tumor is characterized by cells having an increased glycogen expression or activity relative to corresponding non-tumor cells of similar origin.

Another aspect of the present disclosure is directed to a combination therapy. The combination therapy includes an inhibitor of glycogen metabolism, and a cancer therapeutic.

Anaplastic thyroid cancer (ATC) is one of the most lethal solid tumors, yet there are no effective, long-lasting treatments for ATC patients. Most tumors, including tumors of the endocrine system, exhibit an increased consumption of glucose to fuel cancer progression, and some cancers meet this high glucose requirement by metabolizing glycogen. It was recently hypothesized that thyroid cancer may utilize glycogen metabolism for tumor progression based on RNA-sequencing data and publicly available databases. Davidson et al., “Thyroid Hormone Receptor Beta Inhibits PI3kAakt-mTOR Signaling Axis in Anaplastic Thyroid Cancer Via Genomic Mechanisms,” Journal of the Endocrine Society 5(8):bvab 102 (2021); Davidson, C. D., and Carr, F. E., “Review of Pharmacological Inhibition of Thyroid Cancer Metabolism,” Journal of Cancer Metastasis and Treatment 7:45 (2021); and Davidson et al., “Thyroid Hormone Receptor Beta as Tumor Suppressor: Untapped Potential in Treatment and Diagnostics in Solid Tumors,” Cancers (Basel) 13:4254 (2021), all of which are hereby incorporated by reference in their entirety. Therefore, the goal was to investigate the presence and role of glycogen in human thyroid tissues and cell lines and to determine the therapeutic potential of inhibiting glycogen metabolism.

Our goal was to determine if ATC cells metabolize glycogen and if this could be exploited for treatment. Glycogen synthase and glycogen phosphorylase (PYG) isoforms were detected in normal thyroid and thyroid cancer cell lines and patient-derived biopsy samples. Inhibition of PYG using CP-91,149 induced apoptosis in ATC cells but not normal thyroid cells. CP-91,149 decreased NADPH levels and induced reactive oxygen species accumulation. CP-91,149 severely blunted ATC tumor growth in vivo. The work establishes glycogen metabolism as a novel metabolic process in thyroid cells that presents a unique, oncogenic target that could offer an improved clinical outcome.

### BRIEF DESCRIPTION OF THE DRAWINGS

FIGS. 1A-1I show that glycogen phosphorylase brain isoform overexpression drives glycogen breakdown in thyroid adenoma and thyroid cancers. FIGS. 1A-1C show patient thyroid tissue microarrays that were probed with antibodies for GYS1 (FIG. 1A), PYGL (FIG. 1B), and PYGB (FIG. 1C) and stained with DAPI. Magnification=20×, scale bar=50 μm. FIGS. 1D-1F show that mean fluorescence intensity was calculated from each tissue core. FIG. 1G shows that patient thyroid tissue microarrays were stained for glycogen content with PAS stain. Magnification=4×, scale bar=500 μm. FIG. 1H shows PAS stain that was quantified as stain intensity per relative core area. FIG. 1I shows PAS staining intensity that was plotted as a logarithmic function with glycogen phosphorylase expression. N=11-12 for each type of thyroid classification. Data are presented as mean #SD. p values were calculated using one-way ANOVA followed by Dunnett's multiple comparison test. ns, no significance; *, p<0.05; **, p≤0.01; ****, p≤0.0001.

FIGS. 2A-2I show representative cell lines of normal thyroid and thyroid cancer cells metabolize glycogen through expression of glycogen synthase and glycogen phosphorylase isozymes. FIGS. 2A-2C show that normal thyroid and thyroid cancer cell lines express GYS1 (FIG. 2A), PYGL (FIG. 2B), and PYGB (FIG. 2C) as determined by RT-qPCR. In FIG. 2D, thyroid cell lines were profiled for protein expression of phosphorylated GYS1, total GYS1, PYGL, and PYGB. In FIG. 2E, glycogen content was measured in each thyroid cell line via colorimetry. In FIGS. 2F-2I, transmission electron microscopy revealed glycogen deposits in Nthy-ori-3-1 (FIG. 2F) and 8505C cells (FIG. 2H) that were reduced via overnight glucose starvation (FIGS. 2G and 2I). Magnification=5000×, scale bar=1 μm. Data are presented as mean±SD. p values were calculated using one-way ANOVA followed by Dunnett's multiple comparison test. *, p<0.05; ***, p≤0.001; ****, p≤0.0001.

FIGS. 3A-3U show that glycogen phosphorylase inhibition increases glycogen content to limit cell viability and proliferation and induce apoptosis in ATC cells. In FIGS. 3A-3E, CP-91,149 (50 μM, 24-hour incubation) increased glycogen content in Nthy-ori-3-1 and ATC cells as shown by colorimetry (FIG. 3A) and TEM (FIGS. 3B-3E). Magnification=5000×, scale bar=1 μm. In FIG. 3F, thyroid cells exhibit a concentration-dependent decrease in cell viability to CP-91,149 (48-hour incubation). FIGS. 3G-3H show that CP-911,49 induces apoptosis and inhibits proliferation in 8505C (FIG. 3G) but not Nthy-ori-3-1 cells (FIG. 3H) as shown via cleaved PARP, cleaved caspase 9 and 7, and Ki-67 levels. FIGS. 3I-3P show quantification of immunoblots for apoptosis markers and Ki-67. FIG. 3Q shows that knockdown of PYGB is sufficient to induce glycogen buildup in 8505C cells. FIG. 3R shows that 8505C cell viability is reduced following three days of PYGB knockdown. FIGS. 3S-3U show that CP-91,149 inhibits 8505C thyrosphere growth at day 3 (FIG. 3U) compared to day 1 (FIG. 3T). Magnification=40×, scale bar=100 μm. Data are presented as mean±SD. p values were calculated using two-way ANOVA followed by a Tukey's multiple comparison test for multigroup analysis, one-way ANOVA followed by Dunnett's multiple comparison test was used for single group analysis, and Student's t test was used for paired comparisons. ns, no significance; *, p<0.05; **, p≤0.01; ****, p≤0.0001.

FIGS. 4A-4P show that CP-91, 149 triggers glucose flux in ATC cells to fuel glycolysis but inhibits NADPH production, increases levels of reactive oxygen species, and limits oxidative phosphorylation. FIGS. 4A-4B show that CP-91,149 causes a buildup in glucose-1-phosphate (FIG. 4A) and a reduction in glucose-6-phosphate (FIG. 4B). FIGS. 4C-4E show that CP-91,149 enhances extracellular acidification rate (FIGS. 4C-4D) and lactate production (FIG. 4E). FIGS. 4F-4H show that CP-91, 149 treatment increases the expression of SLC2A1 (GLUT1) (FIG. 4F), SLC2A3 (GLUT3) (FIG. 4G), and SLC2A4 (GLUT4) (FIG. 4H). FIG. 4I shows that CP-91,149 causes an increase in glucose uptake from the cell culture media. FIGS. 4J-4K show that CP-91,149 decreases the available supply of NADPH (FIG. 4J) and decreases the relative amount of reduced glutathione (GSH) (FIG. 4K). FIG. 4L shows that CP-91,149 enhances the relative levels of reactive oxygen species which can be attenuated with exogenous N-acetyl cysteine or NADPH. FIG. 4M shows that exogenous N-acetyl cysteine, NADPH, and MN-TMP can rescue ATC cells from CP-mediated loss in cell viability. FIGS. 4N-4O show that CP-91,149 decreases the basal oxygen consumption rate in ATC cells. FIG. 4P shows that CP-91,149 treatment decreases the levels of ATP in ATC cells. 8505C cells were incubated with 50 μM CP-91,149 for 24 hours prior to all experiments. Data are presented as mean±SD. p values were calculated using two-way ANOVA followed by Tukey's or Šidák's multiple comparison test as appropriate for multigroup analysis, one-way ANOVA followed by Dunnett's or Tukey's multiple comparison test as appropriate for single group analysis, and Student's t test was used for paired comparisons. ns, no significance; *, p<0.05; **, p≤0.01; ***, p≤0.001****, p≤0.0001.

FIGS. 5A-5P shows that CP-91,149 exhibits drug synergy with inhibitors of MAPK signaling and the pentose phosphate pathway. FIGS. 5A-P show that ATC cell viability was determined with CP-91,149 alone and in combination with sorafenib (FIGS. 5A-5B), lenvatinib (FIGS. 5C-5D), buparlisib (FIGS. 5E-5F), palbociclib (FIGS. 5G-5H), doxorubicin (FIGS. 5I-5J), 2-deoxy-glucose (FIGS. 5K-5L), 3-bromopyruvic acid (FIGS. 5M-5N), and 6-aminonicotinamide (FIGS. 5O-5P) to calculate coefficient of drug interaction scores. Data are presented as mean±SD. CDI values and significance are described in the methods section.

FIGS. 6A-6I show that CP-91,149 induces apoptosis in vivo to restrict ATC tumor growth. In FIG. 6A, nude mice were injected subcutaneously with 8505C cells on day 0, and tumors were allowed to grow for 9 days when mice were reassigned cages to achieve approximately equal weights in each treatment group. Mice were injected daily for 22 days beginning on day 10 (dashed line) with vehicle control, 10 mg/kg sorafenib, 50 mg/kg CP-91,149, or a combination of sorafenib and CP. FIG. 6B shows relative area under the curve for each treatment group. In FIG. 6C, mice were sacrificed on day 33 and tumors were excised and weighed. FIG. 6D shows representative image of an excised tumor from each treatment group. FIG. 6E shows mice that were weighed three times a week following drug injections to monitor toxicity. FIG. 6F shows representative images of tumors stained for Ki-67, cleaved PARP, and glycogen. 20×, scale bar=150 μM for IF. 4×, scale bar=500 μM for PAS. FIG. 6G shows quantification of Ki-67 fluorescent intensity. FIG. 6H shows quantification of cleaved PARP fluorescent intensity. FIG. 6I shows quantification of percent of sample area stained with PAS. Data are presented as mean±SEM. p values were calculated using one-way ANOVA followed by Dunnett's multiple comparison test. ns, no significance; *, p<0.05; **, p≤0.01; ***, p≤0.001****, p≤0.0001.

FIG. 7 is a schematic illustration showing that glycogen phosphorylase inhibition inhibits the pentose phosphate pathway in ATC cells to induce ROS accumulation, mitochondrial dysfunction, and apoptosis. CP-91,149 treatment causes a buildup of glycogen and the glycogen intermediate glucose-1-phosphate. Glucose transporter expression is increased, causing increased glucose flux into the cell to power glycolysis and lactate production and export. The pentose phosphate pathway is impaired to glycogenolysis inhibition, causing a decrease in levels of NADPH and reduced glutathione (GSH). This lack of intracellular reducing power causes an increase in reactive oxygen species, a decrease in the rate of oxygen consumption, decreased ATP levels, and induction of apoptosis through caspase 9, caspase 7, and PARP. CP-91,149 displays drug synergy with the BRAF inhibitor sorafenib.

FIGS. 8A-8F show that CP-91,149 decreases 8505C cell viability in a time-dependent manner and inhibits cell attachment. In FIG. 8A, 8505C cells were treated with CP-91,149 at various concentrations and time points for SRB assay. In FIG. 8B, 8505C were stained with trypan blue and hand counted for dead and alive cells after 48 hours of CP-91,149 incubation at the indicated concentrations. In FIGS. 8C-8D, cells were imaged 48 hours following vehicle (C) or CP treatment (D). Magnification=40×, scale bar=100 μM. In FIG. 8E, cells were challenged to adhere to fibronectin-coated cell culture plates following 24 hours of 50 μM CP treatment, as measured by SRB assay. FIG. 8F shows area under the curve analysis of cell attachment assay. Data are presented as mean±SD. p values were calculated using one-way ANOVA followed by Dunnett's multiple comparison test for single group analysis, and Student's t test was used for paired comparisons. ns, no significance; *, p<0.05; p≤0.01; ***, p≤0.001****, p≤0.0001.

FIGS. 9A-9C show validation of PYGB Knockdown. In FIGS. 9A-9B, 8505C cells were reverse transfected using lipofectamine with antisense PYGB siRNA at the indicated concentrations for 72 hours for RT-qPCR analysis of PYGB expression (FIG. 9A) and 96 hours for immunoblot analysis of PYGB protein (FIG. 9B). FIG. 9C shows densitometry analysis of PYGB immunoblot. Data are presented as mean±SD. p values were calculated using one-way ANOVA followed by Dunnett's multiple comparison test for single group analysis. *, p<0.05; p≤0.01; ***, p≤0.001.

FIGS. 10A-10I show that glycogen metabolism is inhibited with various small molecule inhibitors in ATC cells. In FIGS. 10A-10C, cell viability was determined via SRB assay (FIGS. 10A-10B) and hand counting with trypan blue (FIG. 10C) following treatment with CP316819 at the indicated concentrations for 48 hours. In FIG. 10D, glycogen assay was conducted in 8505C cells following overnight treatment with CP-316819. In FIGS. 10E-10F, cell viability was determined via SRB assay in 8505C (FIG. 10E) and OCUT-2 (FIG. 10F) cells following treatment with guaiacol at the indicated concentrations for 48 hours. In FIG. 10G, glycogen assay was conducted in 8505C cells following overnight treatment with guaiacol. In FIG. 10H, cell viability was determined via SRB assay in 8505C following treatment with yGsy2p-IN-1 at the indicated concentrations for 48 hours. In FIG. 10I, glycogen assay was conducted in 8505C cells following overnight treatment with yGsy2p-IN-1. Data are presented as mean±SD. p values were calculated using one-way ANOVA followed by Dunnett's multiple comparison test for single group analysis, and Student's t test was used for paired comparisons. ns, no significance; *, p<0.05; **, p≤0.01; ***, p≤0.001****, p≤0.0001.

FIGS. 11A-11C show baseline transcript levels of glucose transporters in thyroid cancer cell lines. In FIGS. 11A-11C, thyroid cells were cultured in normal media conditions and analyzed for expression of SLC2A1 (FIG. 11A), SLC2A3 (FIG. 11B), and SLC2A4 (FIG. 11C) using RT-qPCR. Data are presented as mean±SD. p values were calculated using one-way ANOVA followed by Dunnett's multiple comparison test for single group analysis. *, p<0.05; p≤0.01; ***, p≤0.001****, p<0.0001.

FIGS. 12A-12D show that glucose and pyruvate availability selectively modulate ATC cell susceptibility to glycolytic and glycogenolytic inhibition. In FIG. 12A, 8505C cells were treated with CP or 2-DG for 48 hours in the indicated cell culture media, and cell viability was determined via SRB assay. In FIG. 12B, 8505C cells were treated with CP or 2-DG in the indicated cell culture media prior to ROS determination. In FIG. 12C, 8505C cells were treated with NADPH, 2-DG, or both immediately prior to ROS assay in replete media. In FIG. 12D, 8505C cells were treated with NADPH, 2-DG, or both for 48 hours in replete media prior to SRB assay. Data are presented as mean±SD. p values were calculated using two-way ANOVA followed by Tukey's multiple comparison test for multigroup analysis and one-way ANOVA followed by Dunnett's or Tukey's multiple comparison test as appropriate for single group analysis. ns, no significance; *, p<0.05; **, p≤0.01; ***, p≤0.001****, p≤0.0001.

FIGS. 13A-13B show that sorafenib is synergistic with inhibition of the PPP but not glycolysis. In FIGS. 13A-13B, SRB assay was conducted on 8505C cells treated with the indicated combinations of inhibitors for 48 hours (FIG. 13A) to calculate CDI values (FIG. 13B). Data are presented as mean±SD. CDI values and significance are described in the methods section.

### DETAILED DESCRIPTION

The present disclosure is directed to the use of glycogen metabolism inhibitors as a neo-adjuvant or adjuvant therapy for early stage, aggressive, and treatment-resistant disease. As described herein, glycogen metabolism inhibitors applied to cancer cells induce tumor suppressive transcriptome changes that induce or enhance cancer cell responsiveness to treatment with selective intracellular signaling inhibitors.

Accordingly, a first aspect of the present disclosure relates to a method of treating cancer in a subject. The method includes administering to a subject having a cancer an inhibitor of glycogen metabolism under conditions effective to treat the cancer, wherein the cancer is characterized by cells having an increased glycogen expression or activity relative to corresponding non-cancer cells of similar origin.

The inhibitor of glycogen metabolism described herein includes any compound that is capable of inhibiting, reducing, modulating, or eliminating glycogen metabolism. Examples of suitable inhibitors of glycogen metabolism include those known in the art (see, e.g., Zois et al., “Glycogen Metabolism has a Key Role in the Cancer Microenvironment and Provides New Targets for Cancer Therapy,” J. Mol. Med. 94:137-154 (2016), which is hereby incorporated by reference in its entirety). Exemplary modulators of glycogen metabolism include, for example and without limitation, metformin, lithium, valproate, sodium tungstate, and dichloroacetate. In some embodiments, the inhibitor of glycogen metabolism is selected from sodium tungstate, metformin, lithium, valproate, dichloroacetate, or any combination thereof.

Suitable inhibitors of glycogen metabolism include inhibitors of glycogen phosphorylase, which is a key enzyme driving glycogenolysis. Glycogen phosphorylase inhibitors may be classified by their site of action, e.g., catalytic active site inhibitors, nucleotide binding site adenosine monophosphate (AMP) site inhibitors, purine nucleotide site inhibitors, and indole site inhibitors. In some embodiments, the inhibitor of glycogen metabolism is an inhibitor of glycogen phosphorylase.

Suitable inhibitors of glycogen phosphorylase that work by inhibiting the catalytic active site include, without limitation, glucose analogs, such as N-acetyl-β-D-glucosamine and glucopyranose spirohydantoin, and the azasugar 1,4-dideoxy-1,4-amino-D-arabinitol (DAB) (Andersen et al., “Inhibition of Glycogenolysis in Primary Rat Hepatocytes by 1,4-dideoxy-1,4-imino-D-arabinitol,” Biochem. J. 342(Pt 3):545-50 (1999) and Jakobsen et al., “Iminosugars: Potential Inhibitors of Liver Glycogen Phosphorylase,” Bioorg. Med. Chem. 9:733-44 (2001), both which are hereby incorporated by reference in their entirety).

Exemplary glycogen phosphorylase inhibitors that bind to the AMP site, include, without limitation, 5-chloro-N-[(2S,3R)-4-(dimethylamino)-3-hydroxy-4-oxo-1-phenylbutan-2-yl]-1H-indole-2-carboxamide (CP-91149), 5-chloro-N-[(2S,3R)-4-[(3R,4S)-3,4-dihydroxypyrrolidin-1-yl]-3-hydroxy-4-oxo-1-phenylbutan-2-yl]-1H-indole-2-carboxamide (ingliforib), 5-chloro-N-[(2S)-3-(4-fluorophenyl)-1-(4-hydroxypiperidin-1-yl)-1-oxopropan-2-yl]-1H-indole-2-carboxamide (CP-320626), (2R,3S)-2,3-bis[[(E)-3-(4-hydroxyphenyl) prop-2-enoyl]oxy]pentanedioic acid (FR258900), N-(3,5-dimethyl-benzoyl)-N′-(β-D-glucopyranosyl) urea (KB228), 5-chloro-N-[(2S,3R)-3-hydroxy-4-[methoxy(methyl)amino]-4-oxo-1-phenylbutan-2-yl]-1H-indole-2-carboxamide (CP-316819), 5-chloro-N-[3-(4-fluorophenyl)-1-(4-hydroxypiperidin-1-yl)-1-oxopropan-2-yl]-1H-indole-2-carboxamide (CP320626), isopropyl 4-(2-chlorophenyl)-1-ethyl-2-methyl-5-oxo-1,4,5,7-tetrahydro-furo[3,4-b]pyridine-3-carboxylate (BAY R3401), (4S)-1-ethyl-6-methyl-4-phenyl-5-propan-2-yloxycarbonyl-4H-pyridine-2,3-dicarboxylic acid (BAY-W1807), 1,4-dideoxy-1,4-amino-D-arabinitol (DAB), 4-[3-(2-Chloro-4,5-Difluoro-Benzoyl) ureido]-3-Trifluoromethoxybenzoic Acid (AVE5688), GSK1362885, PSN-357, or any combination thereof. Exemplary glycogen phosphorylase purine nucleoside site inhibitors suitable for use in the methods and combination therapy as described herein include, without limitation, purines, flavopiridol, nucleosides, and olefin derivatives of flavopiridol.

Glycogen phosphorylase inhibitors suitable for inclusion in the methods and combination therapy of the present disclosure may include, without limitation, (2R,3S)-2,3-bis[[(E)-3-(4-hydroxyphenyl) prop-2-enoyl]oxy]pentanedioic acid (FR258900), N-(3,5-dimethyl-benzoyl)-N′-(β-D-glucopyranosyl) urea (KB228), 5-chloro-N-[(2S,3R)-3-hydroxy-4-[methoxy(methyl)amino]-4-oxo-1-phenylbutan-2-yl]-1H-indole-2-carboxamide (CP-316819), 4-[3-(2-Chloro-4,5-Difluoro-Benzoyl) ureido]-3-Trifluoromethoxybenzoic Acid (AVE5688), GSK1362885, and PSN-357.

In some embodiments, the inhibitor of glycogen metabolism is an indole carboxamide site inhibitor of glycogen phosphorylase, e.g., 5-chloro-N-[(2S,3R)-4-(dimethylamino)-3-hydroxy-4-oxo-1-phenylbutan-2-yl]-1H-indole-2-carboxamide (CP-91149).

The inhibitor of glycogen metabolism described herein is contemplated, in some embodiments, to be administered alone in the methods described herein. In some embodiments, no additional therapy is administered beyond the inhibitor of glycogen metabolism.

The inhibitor of glycogen metabolism referred to herein may include the initial treatment given to a patient based upon the diagnosis of cancer in the patient. The diagnosis of cancer may be the first occurrence of that disease in the patient, i.e., a newly diagnosed patient, or a reoccurrence of the disease in a patient, i.e., a relapsed patient. The inhibitor of glycogen metabolism may be part of an initial set of treatments. When used by itself, the inhibitor of glycogen metabolism may be referred to as first-line therapy. Accordingly, in some embodiments, the methods and combination therapy described herein contemplate the administration of one or more additional cancer therapies which are described in detail infra.

The methods described herein may further include, in some embodiments, administering of an additional cancer therapy. The additional cancer therapy or cancer therapeutic (interchangeably referred to herein as a “primary cancer therapy” and/or “primary cancer therapeutic”) may be any therapy or therapeutic suitable for the treatment of cancer, such as a solid malignant tumor. The additional cancer therapy as described herein comprises a cancer therapeutic. The term “cancer therapeutic” may refer to the initial treatment given to a patient based upon the diagnosis of cancer in the patient. The cancer therapeutic may be part of a standard set of treatments and may be applied to a newly diagnosed patient, or a relapsed patient. If it does not cure the disease, slow disease progression, or if it causes severe side effects, other treatment may be added or used instead.

In some embodiments, the additional cancer therapy may be selected from any one or more of MAPK inhibitor, a pentose phosphate pathway inhibitor, an inhibitor of cancer stem cell formation, a cyclin dependent kinase (CDK) inhibitor, a tyrosine kinase inhibitor, a thyroid hormone receptor beta-1 (TRβ) agonist, an anthracycline antibiotic, a glucose molecule, a pyruvic acid derivative, a phosphogluconate dehydrogenase inhibitor, an activator of Interferon/JAK1/STAT1 signaling, a phosphoinositide 3-kinase (PI3K) inhibitor, a PTEN activator, an anti-estrogen, or any combination thereof.

In some embodiments, the primary therapeutic of the combination therapy disclosed herein is an inhibitor of the mitogen-activated protein kinase (MAPK) signaling pathway. The MAPK signaling pathway is a complex signaling pathway that is initiated by an extracellular stimulus in the form of growth factor(s) binding and activating receptor tyrosine kinases on the cell membrane. Downstream activation of RAS, RAF, and MEK proteins converge in the activation of ERK1/2 transcription factor activator. In some embodiments, the MAPK inhibitor is a RAS inhibitor, in particular, a KRAS inhibitor. In some embodiments, the MAPK inhibitor is a RAF inhibitor, in particular, a BRAF inhibitor. In some embodiments, the MAPK inhibitor is a MEK inhibitor. In some embodiments, the MAPK inhibitor is an ERK inhibitor.

MAPK inhibitors currently in use or in development for the treatment of cancer can be utilized as the primary therapeutic in the combination therapy as disclosed herein (see e.g., Braicu et al., “A Comprehensive Review on MAPK: A Promising Therapeutic Target in Cancer,” Cancers 11:1618 (2019), which is hereby incorporated by reference in its entirety. Non-limiting examples of MAPK inhibitors and their target of inhibition are summarized in Table 1 below.

TABLE 1
MAPK Inhibitors

| Tradename | IUPAC Name | Target of Inhibition |
|---|---|---|
| AMG-510 | 6-fluoro-7-(2-fluoro-6-hydroxyphenyl)-1-(4- | KRAS inhibitor |
| | methyl-2-propan-2-ylpyridin-3-yl)-4-[(2S)-2- | |
| | methyl-4-prop-2-enoylpiperazin-1-yl]pyrido[2,3- | |
| | d]pyrimidin-2-one | |
| MRTX849 | 2-[(2S)-4-[7-(8-chloronaphthalen-1-yl)-2-[(2S)-1- | KRAS inhibitor |
| | methylpyrrolidin-2-yl]methoxy]-6,8-dihydro-5H- | |
| | pyrido[3,4-d]pyrimidin-4-yl]-1-(2-fluoroprop-2- | |
| | enoyl)piperazin-2-yl]acetonitrile | |
| sorafenib | 4-[4-[[4-chloro-3- | BRAF inhibitor |
| | (trifluoromethyl)phenyl]carbamoylamino]phenoxy]- | |
| | N-methylpyridine-2-carboxamide | |
| vemurafenib | N-[3-[5-(4-chlorophenyl)-1H-pyrrolo[2,3- | BRAF inhibitor |
| | b]pyridine-3-carbonyl]-2,4-difluorophenyl]propane- | |
| | 1-sulfonamide | |
| dabrafenib | N-[3-[5-(2-aminopyrimidin-4-yl)-2-tert-butyl-1,3- | BRAF inhibitor |
| | thiazol-4-yl]-2-fluorophenyl]-2,6- | |
| | difluorobenzenesulfonamide | |
| selumetinib | 6-(4-bromo-2-chloroanilino)-7-fluoro-N-(2- | MEK inhibitor |
| | hydroxyethoxy)-3-methylbenzimidazole-5- | |
| | carboxamide | |
| trametinib | N-[3-[3-cyclopropyl-5-(2-fluoro-4-iodoanilino)-6,8- | MEEK 1,2 inhibitor |
| | dimethyl-2,4,7-trioxopyrido[4,3-d]pyrimidin-1- | |
| | yl]phenyl]acetamide | |
| PD184352 | 2-(2-chloro-4-iodoanilino)-N- | MEK inhibitor |
| | (cyclopropylmethoxy)-3,4-difluorobenzamide | |
| Pimasertib | N-[(2S)-2,3-dihydroxypropyl]-3-(2-fluoro-4- | MEK 1,2 inhibitor |
| | iodoanilino)pyridine-4-carboxamide | |
| binimetinib | 6-(4-bromo-2-fluoroanilino)-7-fluoro-N-(2- | MEK 1,2 inhibitor |
| | hydroxyethoxy)-3-methylbenzimidazole-5- | |
| | carboxamide | |
| cobimetinib | [3,4-difluoro-2-(2-fluoro-4-iodoanilino)phenyl]-[3- | MEK 1 inhibitor |
| | hydroxy-3-[(2S)-piperidin-2-yl]azetidin-1- | |
| | yl]methanone | |
| ulixertinib | N-[(1S)-1-(3-chlorophenyl)-2-hydroxyethyl]-4-[5- | ERK 1,2 inhibitor |
| | chloro-2-(propan-2-ylamino)pyridin-4-yl]-1H- | |
| | pyrrole-2-carboxamide | |
| silymarin | (2R,3R)-3,5,7-trihydroxy-2-[(2R,3R)-3-(4-hydroxy- | ERK inhibitor |
| | 3-methoxyphenyl)-2-(hydroxymethyl)-2,3-dihydro- | |
| | 1,4-benzodioxin-6-yl]-2,3-dihydrochromen-4-one | |

In some embodiments, when the MAPK inhibitor is a KRAS inhibitor, the KRAS inhibitor is selected from AMG-510, MRTX849, or a combination thereof. In some embodiments, when the MAPK inhibitor is a BRAF inhibitor, the BRAF inhibitor is selected from sorafenib, vemurafenib, dabrafenib, or a combination thereof. In some embodiments, when the MAPK inhibitor is a MEK inhibitor, the MEK inhibitor selected from selumetinib, trametinib, or a combination thereof. In some embodiments, when the MAPK inhibitor is an ERK inhibitor, the ERK inhibitor is selected from ulixertinib, silymarin (rapamycin), or a combination thereof.

In some embodiments, when the additional cancer therapy is a pentose phosphate pathway inhibitor, the pentose phosphate pathway inhibitor is selected from a glucose-6-phosphate dehydrogenase (G6PDH) inhibitor, 6-aminonicotinamide (6-AN), 4-((5-oxo-6,7,8,9-tetrahydro-5H-cyclohepta[d]pyrimidin-2-yl)amino)thiophene-2-carbonitrile (G6PDi-1), (2S,3R,4S,5S,6R)-2-[3-hydroxy-5-[(E)-2-(4-hydroxyphenyl) ethenyl]phenoxy]-6-(hydroxymethyl) oxane-3,4,5-triol (polydatin), a 6-phosphogluconate dehydrogenase (6-PGD) inhibitor, 1,8-dihydroxy-3-methoxy-6-methylanthracene-9,10-dione (physcion), 2-phenyl-1,2-benzoselenazol-3-one (ebselen), or a combination thereof.

In some embodiments, the additional cancer therapy referred to in the methods and combination therapy described herein is a cyclin dependent kinase (CDK) inhibitor. In some embodiments, the CDK inhibitor is a pan-CDK inhibitor. In some embodiments, the CDK inhibitor is a CDK4/6 inhibitor. In some embodiments, the CDK inhibitor is an inhibitor of CDK2, CDK5, CDK7, CDK8, CDK9, CDK12, or combinations thereof. Exemplary CDK inhibitors for inclusion in the combination therapy are known in the art (see, e.g., Sanchez-Martinez et al., “Cyclin Dependent Kinase (CDK) Inhibitors as Anticancer Drugs: Recent Advances (2015-2019),” Bioorganic & Medicinal Chemistry Letters 29:126637 (2019), which is hereby incorporated by reference in its entirety) and summarized in Table 2 below.

TABLE 2
Small Molecule CDK Inhibitors

| Tradename | IUPAC Name/Chemical Structure | Target CDK |
|---|---|---|
| palbociclib | 6-acetyl-8-cyclopentyl-5-methyl-2-[(5- | CDK4/6 |
| | piperazin-1-ylpyridin-2-yl)amino]pyrido[6,5- | |
| | d]pyrimidin-7-one | |
| ribociclib | 7-cyclopentyl-N,N-dimethyl-2-{[5-(piperazin- | CDK4/6 |
| | 1-yl)pyridin-2-yl]amino}-7H-pyrrolo[2,3- | |
| | d]pyrimidine-6-carboxamide | |
| Abemaciclib | N-[5-[(4-ethylpiperazin-1-yl)methyl]pyridin- | CDK4/6 |
| | 2-yl]-5-fluoro-4-(7-fluoro-2-methyl-3-propan- | |
| | 2-ylbenzimidazol-5-yl)pyrimidin-2-amine | |
| Trilaciclib | 2-[[5-(4-methylpiperazin-1-yl)pyridin-2- | CDK4/6 |
| | yl]amino]spiro[7,8- | |
| | dihydropyrazino[5,6]pyrrolo[1,2- | |
| | d]pyrimidine-9,1′-cyclohexane]-6-one | |
| SHR-6390 | 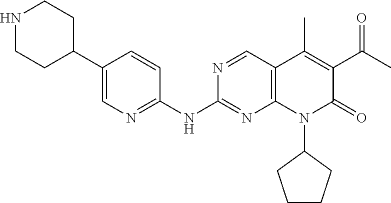 [SMILES: CC(=O)c1c(C)c2cnc(Nc3ccc(C4CCNCC4)cn3)nc2n(C2CCCC2)c1=O] | CDK4/6 |
| Milciclib | N,1,4,4-tetramethyl-8-[4-(4-methylpiperazin- | CDK2 |
| | 1-yl)anilino]-5H-pyrazolo[4,3-h]quinazoline- | |
| | 3-carboxamide | |
| BCD-115 | | CDK8/19 |
| MM-D37K | Synthetic peptide inhibitor | CDK4/6 |
| PF-06873600 | 6-(difluoromethyl)-8-[(1R,2R)-2-hydroxy-2- | CDK2/4/6 |
| | methylcyclopentyl]-2-[(1- | |
| | methylsulfonylpiperidin-4- | |
| | yl)amino]pyrido[2,3-d]pyrimidin-7-one | |
| Alvocidib | 2-(2-chlorophenyl)-5,7-dihydroxy-8-[(3S,4R)- | CDK9 |
| | 3-hydroxy-1-methylpiperidin-4-yl]chromen-4- | |
| | one | |
| Fostamatinib | [6-[5-fluoro-2-(3,4,5- | |
| | trimethoxyanilino)pyrimidin-4-yl]amino]-2,2- | |
| | dimethyl-3-oxopyrido[3,2-b][1,4]oxazin-4- | |
| | yl]methyl dihydrogen phosphate | |
| Zotiraciclib | (16E)-14-methyl-20-oxa-5,7,14,27- | CDK9 |
| | tetrazatetracyclo[19.3.1.12,6.18,12]heptacosa- | |
| | 1(25),2(27),3,5,8,10,12(26),16,21,23-decaene | |
| C7001 | (3R,4R)-4-[[[7-(benzylamino)-3-propan-2- | CDK7 |
| (ICEC 0942) | ylpyrazolo[1,5-a]pyrimidin-5- | |
| | yl]amino]methyl]piperidin-3-ol | |
| BEY-1107 | 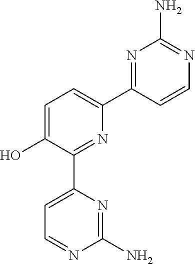 [SMILES: Nc1nccc(-c2ccc(O)c(-c3ccnc(N)n3)n2)n1] | CDK1 |
| BPI-16350 | | CDK4/6 |
| FCN-437 | | CDK4/6 |
| CYC-065 | (2R,3S)-3-[6-[(4,6-dimethylpyridin-3- | CDK2/7/9 |
| | yl)methylamino]-9-propan-2-ylpurin-2- | |
| | yl]amino]pentan-2-ol | |
| Seliciclib | (2R)-2-[6-(benzylamino)-9-propan-2-ylpurin- | CDK2/9 |
| | 2-yl]amino]butan-1-ol | |
| AT-7519 | 4-[(2,6-dichlorobenzoyl)amino]-N-piperidin- | CDK9 |
| | 4-yl-1H-pyrazole-5-carboxamide | |
| Inditinib | 5-Nitro-5′-hydroxy-indirubin-3′-oxime | CDK2 |
| (AGM-130) | | |
| FN-1501 | N-[4-[(4-methylpiperazin-1- | CDK2 |
| | yl)methyl]phenyl]-4-(7H-pyrrolo[2,3- | |
| | d]pyrimidin-4-ylamino)-1H-pyrazole-5- | |
| | carboxamide | |
| SY-1365 | N-[(1S,3R)-3-[[5-chloro-4-(1H-indol-3- | CDK7 |
| | yl)pyrimidin-2-yl]amino]-1- | |
| | methylcyclohexyl]-5-[[(E)-4- | |
| | (dimethylamino)but-2-enoyl]amino]pyridine- | |
| | 2-carboxamide | |
| AZD-4573 | (1S,3R)-3-acetamido-N-[5-chloro-4-(5,5- | CDK9 |
| | dimethyl-4,6-dihydropyrrolo[1,2-b]pyrazol-3- | |
| | yl)pyridin-2-yl]cyclohexane-1-carboxamide | |
| TP-1287 | | CDK9 |
| Voruciclib | 2-[2-chloro-4-(trifluoromethyl)phenyl]-5,7- | CDK9 |
| | dihydroxy-8-[(2R,3S)-2-(hydroxymethyl)-1- | |
| | methylpyrrolidin-3-yl]chromen-4-one | |
| BAY- | 5-fluoro-4-(4-fluoro-2-methoxyphenyl)-N-[4- | CDK9 |
| 1251152 | [(methylsulfonimidoyl)methyl]pyridin-2- | |
| | yl]pyridin-2-amine | |
| Dinaciclib | 2-[(2S)-1-[3-ethyl-7-[(1-oxidopyridin-1-ium- | CDK12 |
| | 3-yl)methylamino]pyrazolo[1,5-a]pyrimidin- | |
| | 5-yl]piperidin-2-yl]ethanol | |

In some embodiments, when the additional cancer therapy is a CDK inhibitor, the CDK inhibitor is selected from 6-acetyl-8-cyclopentyl-5-methyl-2-[(5-piperazin-1-ylpyridin-2-yl)amino]pyrido[6,5-d]pyrimidin-7-one (palbociclib), 7-cyclopentyl-N,N-dimethyl-2-{[5-(piperazin-1-yl)pyridin-2-yl]amino}-7H-pyrrolo[2,3-d]pyrimidine-6-carboxamide (ribociclib), N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (Abemaciclib), 2-[[5-(4-methylpiperazin-1-yl)pyridin-2-yl]amino]spiro[7,8-dihydropyrazino[5,6]pyrrolo[1,2-d]pyrimidine-9,1′-cyclohexane]-6-one (Trilaciclib), Alvocidib, Fostamatinib, or a combination thereof.

In some embodiments, the additional cancer therapy may be a TRβ1 agonist. In some embodiments, the TRβ1 agonist is a selective TRβ1 agonist, exhibiting little or no binding to, or activity at, other thyroid receptor subtypes. In some embodiments, the TRβ1 agonist of the methods and combination therapy does not bind to the TRα1 receptor. In some embodiments, the TRβ1 agonist of the methods and combination therapy does not bind to any of the TRα receptors, i.e., TRα1, TRα2, TRα3. In some embodiments, the TRβ1 agonist of the methods and combination therapy does not bind to other TRβ receptor subtypes, i.e., TRβ2 or TRβ3.

Suitable TRβ1 agonists for inclusion in the methods and combination therapy as described herein include those known in the art. These TRβ1 agonists include, without limitation, 3,5-Dimethyl-4 (4′-hydroxy-3′-isopropylbenzyl) phenoxy) acetic acid (sobetirome; GC-1), 2-{4-[(3-benzyl-4-hydroxyphenyl)methyl]-3,5-dimethylphenoxy}acetic acid (GC-24), 2-[3,5-dichloro-4-[(6-oxo-5-propan-2-yl-1H-pyridazin-3-yl)oxy]phenyl]-3,5-dioxo-1,2,4-triazine-6-carbonitrile (MGL-3196; Resmetirom), 2-[4-(4-hydroxy-3-iodophenoxy)-3,5-diiodophenyl]acetic acid (tiratricol), (2S)-2-amino-3-[4-(4-hydroxy-3-iodophenoxy)-3,5-diiodophenyl]propanoic acid (triiodothyronine; T3), (2R,4S)-4-(3-chlorophenyl)-2-[(3,5-dimethyl-4-(4′-hydroxy-3′-isopropylbenzyl) phenoxy)methyl]-2-oxido-[1-3]-dioxaphosphonane (Mb07811), (2R)-2-amino-3-[4-(4-hydroxy-3,5-diiodophenoxy)-3,5-diiodophenyl]propanoic acid (dextrothyroxine), 2-[3,5-dichloro-4-(4-hydroxy-3-propan-2-ylphenoxy)phenyl] acetic acid (KB-141), 3-[[3,5-dibromo-4-[4-hydroxy-3-(1-methylethyl)-phenoxy]-phenyl]-amino]-3-oxopropanoic acid (KB2115), (2S)-2-amino-3-[4-(4-hydroxy-3,5-diiodophenoxy)-3,5-diiodophenyl]propanoate (2S)-2-amino-3-[4-(4-hydroxy-3-iodophenoxy)-3,5-diiodophenyl]propanoate (Liotrix), (3,5-dimethyl-4-(4′-hydroxy-3′-isopropylbenzyl) phenoxy)methylphosphonic acid (MB07344). Derivatives and analogs of the aforementioned compounds having enhanced selectivity or agonist activity are also suitable for use in the combination therapeutic as described herein. As used herein, the term “a derivative thereof” refers to a salt thereof, a pharmaceutically acceptable salt thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof.

In some embodiments, the TRβ1 agonist of the combination therapy is 2-[4-[(4-hydroxy-3-propan-2-ylphenyl)methyl]-3,5-dimethylphenoxy]acetic acid, which is also known as sobetirome and GC-1 as disclosed in U.S. Pat. No. 5,883,294 to Scanlan et al., which is hereby incorporated by reference in its entirety. In other embodiments, the TRβ1 agonist is a GC-1 derivative as disclosed in U.S. Patent Application Publication No 2016/0244418 to Scanlan et al., which is hereby incorporated by reference in its entirety. In some embodiments, the method and combination therapy described herein does not include a TRβ1 agonist.

In some embodiments, the TRβ1 agonist of the combination therapy is 2-{4-[(3-benzyl-4-hydroxyphenyl)methyl]-3,5-dimethylphenoxy} acetic acid (GC-24), 2-[3,5-dichloro-4-[(6-oxo-5-propan-2-yl-1H-pyridazin-3-yl)oxy]phenyl]-3,5-dioxo-1,2,4-triazine-6-carbonitrile, which is also known as MGL-3196 and Resmetirom, or a derivative thereof, as disclosed in U.S. Pat. No. 7,807,674 to Haynes et al., which is hereby incorporated by reference in its entirety In other embodiments, the TRβ1 agonist is a prodrug of MGL-3196 as disclosed in U.S. Pat. No. 8,076,334 to Haynes, which is hereby incorporated by reference in its entirety.

In some embodiments, the TRβ agonist is selected from 3,5-Dimethyl-4 (4′-hydroxy-3′-isopropylbenzyl) phenoxy) acetic acid (sobetirome; GC-1), 2-{4-[(3-benzyl-4-hydroxyphenyl)methyl]-3,5-dimethylphenoxy} acetic acid (GC-24), MGL-3196 (Resmetirom), 2-[4-(4-hydroxy-3-iodophenoxy)-3,5-diiodophenyl]acetic acid (tiratricol), (2S)-2-amino-3-[4-(4-hydroxy-3-iodophenoxy)-3,5-diiodophenyl]propanoic acid (triiodothyronine; T3), (2R)-2-amino-3-[4-(4-hydroxy-3,5-diiodophenoxy)-3,5-diiodophenyl]propanoic acid (dextrothyroxine), 2-[3,5-dichloro-4-(4-hydroxy-3-propan-2-ylphenoxy)phenyl] acetic acid (KB-141), 3-[[3,5-dibromo-4-[4-hydroxy-3-(1-methylethyl)-phenoxy]-phenyl]-amino]-3-oxopropanoic acid (KB2115), (2S)-2-amino-3-[4-(4-hydroxy-3,5-diiodophenoxy)-3,5-diiodophenyl]propanoate (2S)-2-amino-3-[4-(4-hydroxy-3-iodophenoxy)-3,5-diiodophenyl]propanoate (Liotrix), (3,5-dimethyl-4-(4′-hydroxy-3′-isopropylbenzyl) phenoxy)methylphosphonic acid (MB07344), (2R,4S)-4-(3-chlorophenyl)-2-[(3,5-dimethyl-4-(4′-hydroxy-3′-isopropylbenzyl) phenoxy)methyl]-2-oxido-[1-3]-dioxaphosphonane (Mb07811), or derivatives thereof.

In some embodiments, the additional cancer therapy is a phosphoinositide 3-kinase (PI3K) inhibitor. Suitable PI3K inhibitors include those known in the art. Exemplary PI3K inhibitors include, without limitation, 5-(2,6-dimorpholin-4-ylpyrimidin-4-yl)-4-(trifluoromethyl)pyridin-2-amine (buparlisib), 4-morpholino-2-phenylquinazolines, pyrido[3′,2′:4,5]furo[3,2-d]pyrimidine, pyrido[3′,2′:4,5]furo[3,2-d]pyrimidine, PWT-458 (pegylated-17-hydroxywortmannin), PX-866 (wortmannin analogue), 3-[6-(morpholin-4-yl)-8-oxa-3,5,10-triazatricyclo[7.4.0.0\hat{ }{2,7}]trideca-1(13),2,4,6,9,11-hexaen-4-yl]phenol (PI103), 5-[bis(morpholin-4-yl)-1,3,5-triazin-2-yl]-4-(trifluoromethyl)pyridin-2-amine (PQR-309), (S)-3-(1-((9H-purin-6-yl)amino)ethyl)-8-chloro-2-phenylisoquinolin-1 (2H)-one (Duvelisib), N-[4-[[3-(3,5-dimethoxyanilino) quinoxalin-2-yl]sulfamoyl]phenyl]-3-methoxy-4-methylbenzamide (Voxtalisib), 1-[(3S)-3-[6-[6-methoxy-5-(trifluoromethyl)pyridin-3-yl]-7,8-dihydro-5H-pyrido[4,3-d]pyrimidin-4-yl]amino]pyrrolidin-1-yl]propan-1-one (Leniolisib), (1,1-dimethylpiperidin-1-ium-4-yl) octadecyl phosphate (perifosine), 5-[7-methanesulfonyl-2-(morpholin-4-yl)-5H,6H,7H-pyrrolo[2,3-d]pyrimidin-4-yl]pyrimidin-2-amine (MEN1611), (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib), (1S,3S,4R)-4-[(3aS,4R,5S,7aS)-4-(aminomethyl)-7a-methyl-1-methylidene-3,3a,4,5,6,7-hexahydro-2H-inden-5-yl]-3-(hydroxymethyl)-4-methylcyclohexan-1-ol (rosiptor), 2-[6-(1H-indol-4-yl)-1H-indazol-4-yl]-5-[(4-propan-2-ylpiperazin-1-yl)methyl]-1,3-oxazole (nemiralisib), 2-amino-N-[7-methoxy-8-(3-morpholin-4-ylpropoxy)-2,3-dihydroimidazo[1,2-c]quinazolin-5-yl]pyrimidine-5-carboxamide (copanlisib), 2,4-difluoro-N-[2-methoxy-5-[4-methyl-8-[(3-methyloxetan-3-yl) methoxy]quinazolin-6-yl]pyridin-3-yl]benzenesulfonamide (Compound 5d), [6-(2-amino-1,3-benzoxazol-5-yl) imidazo[1,2-a]pyridin-3-yl]-morpholin-4-ylmethanone (serabelisib), 2-(morpholin-4-yl)-8-phenyl-4H-chromen-4-one (LY294002), 3-(3-fluorophenyl)-2-[(1S)-1-[(9H-purin-6-yl)amino]propyl]-4H-chromen-4-one (tenalisib), (2S)-2-[2-[(4S)-4-(difluoromethyl)-2-oxo-1,3-oxazolidin-3-yl]-5,6-dihydroimidazo[1,2-d][1,4]benzoxazepin-9-yl]amino]propanamide (GDC-0077), 8-(6-methoxypyridin-3-yl)-3-methyl-1-[4-piperazin-1-yl-3-(trifluoromethyl)phenyl]imidazo[5,4-c]quinolin-2-one (BGT 226), [8-[6-amino-5-(trifluoromethyl)pyridin-3-yl]-1-[6-(2-cyanopropan-2-yl)pyridin-3-yl]-3-methylimidazo[4,5-c]quinolin-2-ylidene]cyanamide (panulisib), (5Z)-5-[(4-pyridin-4-ylquinolin-6-yl)methylidene]-1,3-thiazolidine-2,4-dione (GSK1059615), 7-methyl-2-morpholin-4-yl-9-[1-(phenylamino)ethyl]pyrido[2,1-b]pyrimidin-4-one (TGX 221), 4-[6-[[4-(cyclopropylmethyl) piperazin-1-yl]methyl]-2-(5-fluoro-1H-indol-4-yl) thieno[3,2-d]pyrimidin-4-yl]morpholine (PI 3065), 2-(difluoromethyl)-1-[4,6-di(morpholin-4-yl)-1,3,5-triazin-2-yl]benzimidazole (ZSTK474), 1-[4-[4-(dimethylamino) piperidine-1-carbonyl]phenyl]-3-[4-(4,6-dimorpholin-4-yl-1,3,5-triazin-2-yl)phenyl]urea (gedatolisib), 5-fluoro-3-phenyl-2-[(1S)-1-(7H-purin-6-ylamino) propyl]quinazolin-4-one (idelalisib), 3-[2,4-diamino-6-(3-hydroxyphenyl) pteridin-7-yl]phenol (TG-100-115), ethyl 6-[5-(benzenesulfonamido)pyridin-3-yl]imidazo[1,2-a]pyridine-3-carboxylate (HS-173), 2-[2-methoxy-5-[[(E)-2-(2,4,6-trimethoxyphenyl) ethenyl]sulfonylmethyl]phenyl]amino]acetic acid (rigosertib), 2-(6,7-dimethoxyquinazolin-4-yl)-5-pyridin-2-yl-1,2,4-triazol-3-amine (CP-466722), N-[3-(2,1,3-benzothiadiazol-6-ylamino) quinoxalin-2-yl]-4-methylbenzenesulfonamide (pilaralisib), 2-[(1S)-1-[4-amino-3-(3-fluoro-4-propan-2-yloxyphenyl)pyrazolo[3,4-d]pyrimidin-1-yl]ethyl]-6-fluoro-3-(3-fluorophenyl) chromen-4-one (umbralisib), 5-(8-methyl-2-morpholin-4-yl-9-propan-2-ylpurin-6-yl)pyrimidin-2-amine (VS-5584), 2-methyl-2-[4-(3-methyl-2-oxo-8-quinolin-3-ylimidazo[4,5-c]quinolin-1-yl)phenyl]propanenitrile (dactolisib), 1-(4-{5-[5-amino-6-(5-tert-butyl-1,3,4-oxadiazol-2-yl)pyrazin-2-yl]-1-ethyl-1H-1,2,4-triazol-3-yl}piperidin-1-yl)-3-hydroxypropan-1-one (AZD8835), [(3aR,6E,9S,9aR,10R,11aS)-6-[(di(prop-2-enyl)amino)methylidene]-5-hydroxy-9-(methoxymethyl)-9a, 11a-dimethyl-1,4,7-trioxo-2,3,3a,9,10,11-hexahydroindeno[7,6-h]isochromen-10-yl]acetate (sonolisib), 2-[[4-amino-3-(3-fluoro-5-hydroxyphenyl)pyrazolo[3,4-d]pyrimidin-1-yl]methyl]-5-[3-[2-(2-methoxyethoxy) ethoxy]prop-1-ynyl]-3-[2-(trifluoromethyl)phenyl]methyl]quinazolin-4-one (RV-6153), 6-[2-[4-amino-3-(3-hydroxyphenyl)pyrazolo[3,4-d]pyrimidin-1-yl]methyl]-3-[(2-chlorophenyl)methyl]-4-oxoquinazolin-5-yl]-N,N-bis(2-methoxyethyl) hex-5-ynamide (RV-1729), 2-(4-ethylpiperazin-1-yl)-N-[4-(2-morpholin-4-yl-4-oxochromen-8yl)dibenzothiophen-1-yl]acetamide (KU-0060648), N′-cyclopropyl-N-quinolin-6-yl-7H-purine-2,6-diamine (puquitinib), or any combination thereof.

In some embodiments, the additional cancer therapy is an activator of the Interferon/JAK1/STAT1 signaling pathway. STAT1 exhibits tumor suppressor properties in a number of cancers, including colorectal cancer, hepatocellular carcinoma, esophageal cancer, pancreatic cancer, soft tissue sarcoma, and metastatic melanoma. Interferon gamma is a type II interferon that, when bound by cytokine, activates JAK1, which subsequently phosphorylates STAT1, leading to its dimerization, translocation to the nucleus, and activation of its transcription factor activity. Thus, in some embodiments, a suitable activator of the Interferon/JAK1/STAT1 pathway includes, for example and without limitation, a recombinant interferon gamma protein or polypeptides thereof (see e.g., Razaghi et al., “Review of the Recombinant Human Interferon Gamma as an Immunotherapeutic: Impacts of Production Platforms and Glycosylation,” J. Biotech. 240:48-60 (2016), which is hereby incorporated by reference in its entirety). In some embodiments, a suitable activator of the Interferon/JAK1/STAT1 pathway includes a recombinant interferon alpha protein (see e.g., Ningrum R., “Human Interferon Alpha-2b: A Therapeutic Protein for Cancer Treatment,” Scientifica 2014:970315 (2014), which is hereby incorporated by reference in its entirety. Suitable recombinant interferon alpha proteins include, without limitation Intron A® (Schering-Plough) and Roferon-A® (Hoffmann-LaRoche). In some embodiments, the activator of Interferon/JAK1/STAT1 signaling is a recombinant interferon-alpha, recombinant interferon-gamma, or a combination thereof.

In some embodiments, a suitable activator of Interferon/JAK1/STAT1 signaling is recombinant oncostatin M protein (see Schaefer et al., “Activation of Stat3 and Stat1 DNA Binding and Transcriptional Activity in Human Brain Tumour Cell Line by gp130 Cytokine,” Cell Signal 12(3):143-151, which is hereby incorporated by reference in its entirety). In some embodiments, the activator of Interferon/JAK1/STAT1 signaling is selected from recombinant Oncostatin M, IL-6, or a combination thereof. A suitable recombinant oncostatin M protein has an amino acid sequence of SEQ ID NO: 1 as shown below or a polypeptide derived therefrom.

SEQ ID NO: 1
AAIGSCSKEYRVLLGQLQKQTDLMQDTSRLLDPYIRIQGLDVPKLREHC
RERPGAFPSEETLRGLGRRGFLQTLNATLGCVLHRLADLEQRLPKAQDL
ERSGLNIEDLEKLQMARPNILGLRNNIYCMAQLLDNSDTAEPTKAGRGA
SQPPTPTPASDAFQRKLEGCRFLHGYHRFMHSVGRVFSKWGESPNRSR

In some embodiments, a suitable activator of the Interferon/JAK1/STAT1 signaling is a recombinant IL-6 protein. A suitable recombinant IL-6 protein has an amino acid sequence of SEQ ID NO: 2 or polypeptide derived thereof:

SEQ ID NO: 2
VPPGEDSKDVAAPHRQPLTSSERIDKQIRYILDGISALRKETCNKSNMC
ESSKEALAENNLNLPKMAEKDGCFQSGFNEETCLVKIITGLLEFEVYLE
YLQNRFESSEEQARAVQMSTKVLIQFLQKKAKNLDAITTPDPTTNASLL
TKLQAQNQWLQDMTTHLILRSFKEFLQSSLRALRQM

In some embodiments, the additional cancer therapy of the methods and combination therapy as described herein is a PTEN activator. Suitable PTEN activators include those known in the art, see e.g., Boosani and Agrawal, “PTEN Modulators: A Patent Review,” Exp. Opin. Ther. Pat. 23(5):569-80 (2013), which is hereby incorporated by reference in its entirety. In some embodiments, the PTEN activator is an antibody. Suitable PTEN activator antibodies include, without limitation an anti-CD20 antibody (e.g., Ublituximab, Rituximab, and biosimilars thereof) (see e.g., Le Garff-Tavernier et al. “Analysis of CD16+CD56dim NK Cells From CLL Patients: Evidence Supporting a Therapeutic Strategy With Optimized Anti-CD20 Monoclonal Antibodies,” Leukemia 25(1):101-9 (2011), which is hereby incorporated by reference in its entirety), a HER2 antibody (e.g., Trastuzumab, Pertuzumab and biosimilars thereof) (see U.S. Patent application Pub. No. 20020192211 to Hudziak and U.S. Pat. No. 6,399,063 to Hudziak, which are hereby incorporate by reference in their entirety), and an epidermal growth factor receptor antibody (e.g., Cetuximab) (see U.S. Pat. No. 7,060,808 to Goldstein, which is hereby incorporated by reference in its entirety).

In some embodiments, the PTEN activator is small molecule PTEN activator. Suitable small molecule activators of PTEN include, without limitation, N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide N-[2-(diethylamino)ethyl]-5-{[(3Z)-5-fluoro-2-oxo-2,3-dihydro-1H-indol-3-ylidene]methyl}-2,4-dimethyl-1H-pyrrole-3-carboxamide (Sunitinib), N-(3-chloro-4-fluorophenyl)-7-methoxy-6-[3-(morpholin-4-yl) propoxy]quinazolin-4-amine (Gefitinib), N-(3-ethynylphenyl)-6,7-bis(2-methoxyethoxy) quinazolin-4-amine (Erlotinib), [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl]2,2-dimethylbutanoate (Simvastatin), [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl](2S)-2-methylbutanoate (Lovastatin), 5-[4-[2-(methyl-pyridin-2-ylamino) ethoxy]phenyl]methyl]-1,3-thiazolidine-2,4-dione (Rosiglitazone), 7-[3-(azetidin-1-ylmethyl)cyclobutyl]-5-[3-(phenylmethoxy)phenyl]pyrrolo[3,2-e]pyrimidin-4-amine (NVP-AEW541), and (9S,12S,14S,16S,17R)-8,13,14,17-tetramethoxy-4,10,12,16-tetramethyl-3,20,22-trioxo-2-azabicyclo[16.3.1]docosa-1 (21),4,6,10,18-pentaen-9-yl carbamate (Herbimycin), or any combination thereof.

In some embodiments, the additional cancer therapy of the methods and combination therapy described herein is an anti-estrogen therapeutic. Suitable anti-estrogen therapeutics include those known and used in the art. Exemplary anti-estrogen therapeutics include, without limitation, fulvestrant, tamoxifen, clomifene, raloxifene, toremifene, or any combination thereof. Aromatase inhibitors (e.g., Letrozole, Anastrozole, and Exemestane) that lower or reduce the production of estrogen are also suitable primary cancer therapeutics of the combination therapy in some embodiments.

In some embodiments, the additional cancer therapy of the methods and combination therapy described herein is an inhibitor of cancer stem cell formation. In some embodiments the inhibitor of cancer stem cell formation is a tyrosine kinase inhibitor. In some embodiments, the tyrosine kinase inhibitor is a vascular endothelial growth factor (VEGF) inhibitor. Suitable VEGF inhibitors include those known and used in the art, see e.g., those disclosed in Meadows ant Hurwitz, “Anti-VEGF Therapies in the Clinic,” Cold Spring Harb. Perspect. Med. 2:1006577 (2012), which is hereby incorporated by reference in its entirety. In some embodiments, the VEGF inhibitor is a VEGF antibody. Suitable VEGF antibodies include, without limitation, bevacizumab (humanized anti-VEGF monoclonal antibody) and Ranibizumab (monoclonal VEGF-A antibody fragment (Fab)). In some embodiments, the VEGF inhibitor is a recombinant VEGF receptor, such as, for example, Aflibercept, a recombinant VEGF receptor fusion protein that binds VEGF A and B. In some embodiments, the VEGF inhibitor is a small molecule tyrosine kinase inhibitor. Suitable small molecule tyrosine kinase inhibitors include without limitation, the small molecule inhibitors listed in Table 3 below.

TABLE 3
Small Molecule Tyrosine Kinase Inhibitors

| Trade name | IUPAC name | Mode of Action |
|---|---|---|
| Sunitinib | N-[2-(diethylamino)ethyl]-5-[(Z)-(5-fluoro-2-oxo- | Inhibitor of VEGFs, |
| | 1H-indol-3-ylidene)methyl]-2,4-dimethyl-1H- | platelet derived growth |
| | pyrrole-3-carboxamide | factor (PDGF), c-kit, |
| | | Flt3, and Ret |
| Pazopanib | 5-[[4-[(2,3-dimethylindazol-6-yl)- | Inhibitor of VEGFRs, |
| | methylamino]pyrimidin-2-yl]amino]-2- | PDGFR and c-kit |
| | methylbenzenesulfonamide | |
| Axitinib | N-methyl-2-[[3-[(E)-2-pyridin-2-ylethenyl]-1H- | Inhibitor of VEGFs, |
| | indazol-6-yl]sulfany]benzamide | PDGF, and c-kit |
| Regorafenib | 4-[4-[[4-chloro-3- | inhibitor of VEGFRs, |
| | (trifluoromethyl)phenyl]carbamoylamino]-3- | PDGFR, c-kit, Raf, and |
| | fluorophenoxy]-N-methylpyridine-2-carboxamide | Ret |
| Sorafenib | 4-[4-[[4-chloro-3- | Inhibitor of VEGFRs, |
| | (trifluoromethyl)phenyl]carbamoylamino]phenoxy]- | PDGFRs, c-kit, Ret, |
| | N-methylpyridine-2-carboxamide | and Raf |
| Brivanib | [(2R)-1-[4-[(4-fluoro-2-methyl-1H-indol-5-yl)oxy]- | inhibitor of VEGFR-2, |
| alaninate | 5-methylpyrrolo[2,1-f][1,2,4]triazin-6- | PDGFR, and Fibroblast |
| | yl]oxypropan-2-yl] (2S)-2-aminopropanoate | growth factor receptor |
| | | (FGFR) |
| Cediranib | 4-[(4-fluoro-2-methyl-1H-indol-5-yl)oxy]-6- | inhibitor of VEGFs, |
| | methoxy-7-(3-pyrrolidin-1-ylpropoxy)quinazoline | PDGF, and c-kit |
| Vandetanib | N-(4-bromo-2-fluorophenyl)-6-methoxy-7-[(1- | inhibitor of VEGFs, |
| | methylpiperidin-4-yl)methoxy]quinazolin-4-amine | PDGF, and epidermal |
| | | growth factor receptor |
| | | (EGFR), Ret |
| Linifinib | 1-[4-(3-amino-1H-indazol-4-yl)phenyl]-3-(2-fluoro- | inhibitor of VEGFs, |
| | 5-methylphenyl)urea | PDGF, and c-kit |
| Lenvatinib | 4-[3-chloro-4- | inhibitor of VEGFs |
| | (cyclopropylcarbamoylamino)phenoxy]-7- | |
| | methoxyquinoline-6-carboxamide | |
| Nintedanib | methyl 2-hydroxy-3-[N-[4-[methyl-[2-(4- | inhibitor of VEGFs, |
| | methylpiperazin-1-yl)acetyl]amino]phenyl]-C- | PDGFR, and FGFR |
| | phenylcarbonimidoyl]-1H-indole-6-carboxylate | |
| CLM94 | 4-chloro-N-(1,1,3-trioxo-2,3-4- | inhibitor of VEGFR |
| | dihydrobenzo[d]isothiazol-4-yl)benzamide | |
| CLM3 | pyrazolo[3 4-d]pyrimidine derivative | inhibitor of EGFR, Ret, |
| | | VEGFR |
| CLM24 | pyrazolopyricaidine derivative | inhibitor of EGFR, Ret, |
| | | VEGFR |
| CLM29 | pyrazolopyrirnidille derivative | inhibitor of EGFR, Ret, |
| | | VEGFR |

In some embodiments, the tyrosine kinase inhibitor is a receptor tyrosine-protein kinase erbB-2 (also known as HER-2) inhibitor. In some embodiments, the HER2 inhibitor is a HER2 antibody. Suitable HER2 antibodies include, without limitation, the monoclonal antibodies Trastuzumab (Herceptin) and Pertuzumab (Perjeta). In some embodiments, the HER2 inhibitor is an antibody-drug conjugate, such as Ado-trastuzumab emtansine (Kadcyla or TDM-1) and Fam-trastuzumab deruxtecan (Enhertu), which are antibody-chemotherapeutic conjugates. In some embodiments, the HER2 inhibitor is a small molecule inhibitor, such as lapatinib and neratinib.

In some embodiments, the tyrosine kinase inhibitor is an inhibitor of the src family of kinases, including c-Src, Fyn, Yes, Lck, Lyn Hck, Fgr, and Blk, which have all been implicated in the pathogenesis of cancer. Exemplary src family of kinase inhibitors include, but are not limited to imatinib, dasatinib and nilotinib, saracatinib, and bosutinib.

In some embodiments, the additional cancer therapy includes one or more of Sorafenib, Lenvatinib, Bevacizumab, Ranibizumab, Aflibercept, Sunitinib, Pazopanib, Axitinib, Regorafenib, Nintedanib, or any combination thereof.

In accordance with the methods described herein, a “subject” refers to any animal having cancer, where the cancer is characterized by cells having an increased level of glycogen metabolism or activity relative to corresponding non-cancer cells of similar origin.

In some embodiments, the subject is any animal having an advance form of thyroid cancer (e.g., advanced anaplastic thyroid cancer, poorly differentiated thyroid cancer, metastatic thyroid cancer, treatment resistant thyroid cancer, or recurrent thyroid cancer) or an advanced form of breast cancer (e.g., triple negative breast cancer, stage four breast cancer) or breast cancer that is estrogen receptor-positive or otherwise metastatic, HER2 positive breast cancer, or variants thereof. In some embodiments, the subject is a mammal. Exemplary mammalian subjects include, without limitation, humans, non-human primates, dogs, cats, rodents (e.g., mouse, rat, guinea pig), horses, cattle and cows, sheep, and pigs. In some embodiments, the subject does not have or is not suspected of having Diabetes mellitus.

In accordance with the methods described herein, the cancer or cancer cells to be treated include, in some embodiments, any malignant solid tumor or tumor cells, where the cells of the tumor exhibit increased glycogen metabolism relative to corresponding non-cancer cells of similar origin. Suitable cancers and cancer cells to be treated in accordance with the methods described herein include, without limitation, thyroid cancer and thyroid cancer cells, breast cancer and breast cancer cells, colorectal cancer and colorectal cancer cells, bladder cancer and bladder cancer cells, cervical cancer and cervical cancer cells, esophageal cancer and esophageal cancer cells, gastric cancer and gastric cancer cells, head and neck cancer and head and neck cancer cells, kidney cancer and kidney cancer cells, liver cancer and liver cancer cells, lung cancer and lung cancer cells, nasopharyngeal cancer and nasopharyngeal cancer cells, ovarian cancer and ovarian cancer cells, cholangiocarcinoma and cholangiocarcinoma cells, pancreatic cancer and pancreatic cancer cells, prostate cancer and prostate cancer cells, glioblastoma and glioblastoma cells, astrocytoma and astrocytoma cells, melanoma and melanoma cells, mesothelioma, musculoskeletal sarcoma, and soft tissue sarcoma. In another embodiment, the cancer is a blood cancer selected from leukemia, lymphoma, or myeloma. In some embodiments, the cancer described herein includes a population of cancer cells that are differentiated cancer cells. In another embodiment, the cancer described herein includes a population of cancer cells that are undifferentiated or are poorly differentiated cancer cells.

Tumors suitable for treatment in accordance with the methods and combination therapy of the present disclosure are those where the cells of the tumor exhibit increased levels of glycogen metabolism relative to corresponding non-cancer cells of similar origin. Glycogen metabolism “expression levels” encompass the production of any product produced by glycogen including but not limited to transcription of mRNA and translation of polypeptides, peptides, and peptide fragments. Measuring or detecting expression levels encompasses assaying, measuring, quantifying, scoring, or detecting the amount, concentration, or relative abundance of a gene product. It is recognized that methods of assaying glycogen metabolism expression include direct measurements and indirect measurements. One skilled in the art is capable of selecting an appropriate method of evaluating glycogen metabolism levels.

In some embodiments, glycogen metabolism levels are measured using a nucleic acid detection assay. In some embodiments, the DNA levels are measured. In another embodiment, RNA, e.g., mRNA, levels are measured. RNA is preferably reverse-transcribed to synthesize complementary DNA (cDNA), which is then amplified and detected or directly detected. The detected cDNA is measured and the levels of cDNA serve as an indicator of the RNA or mRNA levels present in a sample. Reverse transcription may be performed alone or in combination with an amplification step, e.g., reverse transcription polymerase chain reaction (RT-PCR), which may be further modified to be quantitative, e.g., quantitative RT-PCR as described in U.S. Pat. No. 5,639,606, which is hereby incorporated by reference in its entirety.

In some embodiments, the extracted nucleic acids, including DNA and/or RNA, are analyzed directly without an amplification step. Direct analysis may be performed with different methods including, but not limited to, nanostring technology (Geiss et al. “Direct Multiplexed Measurement of Gene Expression with Color-Coded Probe Pairs,” Nat. Biotechnol. 26(3):317-25 (2008), which is hereby incorporated by reference in its entirety). In some embodiments, direct analysis can be performed using immunohistochemical techniques.

In other embodiments, it may be beneficial or otherwise desirable to amplify the cancer cell extracted nucleic acids prior to detection/analysis. Methods of nucleic acid amplification, including quantitative amplification, are commonly used and generally known in the art. Quantitative amplification will allow quantitative determination of relative amounts of nucleic acids. Nucleic acid amplification methods include, without limitation, polymerase chain reaction (PCR) (U.S. Pat. No. 5,219,727, which is hereby incorporated by reference in its entirety) and its variants such as in situ polymerase chain reaction (U.S. Pat. No. 5,538,871, which is hereby incorporated by reference in its entirety), quantitative polymerase chain reaction (U.S. Pat. No. 5,219,727, which is hereby incorporated by reference in its entirety), nested polymerase chain reaction (U.S. Pat. No. 5,556,773, which is hereby incorporated by reference in its entirety), self-sustained sequence replication and its variants (Guatelli et al. “Isothermal, In vitro Amplification of Nucleic Acids by a Multienzyme Reaction Modeled after Retroviral Replication,” Proc. Natl. Acad. Sci. USA 87(5):1874-8 (1990), which is hereby incorporated by reference in its entirety), transcriptional amplification and its variants (Kwoh et al. “Transcription-based Amplification System and Detection of Amplified Human Immunodeficiency Virus type 1 with a Bead-Based Sandwich Hybridization Format,” Proc. Natl. Acad. Sci. USA 86(4):1173-7 (1989), which is hereby incorporated by reference in its entirety), Qb Replicase and its variants (Miele et al. “Autocatalytic Replication of a Recombinant RNA,” J. Mol. Biol. 171(3):281-95 (1983), which is hereby incorporated by reference in its entirety), cold-PCR (Li et al. “Replacing PCR with COLD-PCR Enriches Variant DNA Sequences and Redefines the Sensitivity of Genetic Testing,” Nat Med 14(5):579-84 (2008), which is hereby incorporated by reference in its entirety) or any other nucleic acid amplification method known in the art. Depending on the amplification technique that is employed, the amplified molecules are detected during amplification (e.g., real-time PCR) or subsequent to amplification using detection techniques known to those of skill in the art. Suitable nucleic acid detection assays include, for example and without limitation, northern blot, microarray, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetric assays, and mass spectrometry (MS) methods (e.g., MassARRAY® System).

In some embodiments, glycogen metabolism levels are measured in the cancer cells. Glycogen metabolism levels can be measured using an immunoassay. Generally, an immunoassay involves contacting the cancer cell sample from the subject with a binding reagent, e.g., an antibody, that binds specifically to the product of glycogen metabolism. The binding reagent is coupled to a detectable label, either directly or indirectly. For example, an antibody can be directly coupled to a detectable label or indirectly coupled to a detectable label via a secondary antibody. The one or more labeled binding reagents bound to the product of glycogen metabolism (i.e., a binding reagent-marker complex) in the sample is detected, and the amount of labeled binding reagent that is detected serves as an indicator of the amount or expression level of glycogen metabolism product(s) in the sample. Immunoassays that are well known in the art and suitable for measuring include, for example and without limitation, an immunohistochemical assay, radioimmunoassay, enzyme linked immunosorbent assay (ELISA), immunoradiometric assay, gel diffusion precipitation reaction, immunodiffusion assay, in situ immunoassay, western blot, precipitation reaction, complement fixation assay, immunofluorescence assay, and immunoelectrophoresis assay.

In some embodiments, protein levels are measured using one-dimensional and two-dimensional electrophoretic gel analysis, high performance liquid chromatography (HPLC), reverse phase HPLC, Fast protein liquid chromatograph (FPLC), mass spectrometry (MS), tandem mass spectrometry, liquid crystal-MS (LC-MS) surface enhanced laser desorption/ionization (SELDI), MALDI, and/or protein sequencing.

In accordance with the methods of the present disclosure, the levels of glycogen metabolism products are compared to glycogen metabolism product levels in non-cancer cells of similar origin, i.e., “control” cells to determine whether the cancer cells will be responsive to the therapeutic treatment and methods described herein. In some embodiments, the control expression level of glycogen metabolism product is the average glycogen metabolism product level of a cell corresponding to the cancerous cell type in a cohort of healthy individuals. In another embodiment, the control glycogen metabolism level is the average glycogen metabolism level in a cell sample taken from the subject to be treated, but at an earlier time point (e.g., a pre-cancerous time point). In all of these embodiments, a decrease in the glycogen metabolism levels in the cancer cells from the subject relative to the control cell expression level identifies the cancer as one suitable for treatment in accordance with the methods described herein.

A “decreased expression level” refers to an expression level (i.e., protein or gene expression level) that is lower than the control level. For example, a decreased expression level is at least 10%, at least 20%, at least 30%, at least 40%, at least 50%, at least 80%, at least 90%, at least 2-fold, at least 3-fold, at least 5-fold, at least 10-fold, at least 50-fold or at least 100-fold lower than the control expression level.

In some embodiments, the methods described herein are used to induce differentiation of thyroid cancer cells for the treatment of thyroid cancer. In some embodiments, the thyroid cancer is advanced anaplastic thyroid cancer, poorly differentiated thyroid cancer, metastatic thyroid cancer, treatment resistant thyroid cancer, or recurrent thyroid cancer.

In some embodiments, the methods described herein are used to induce differentiation in breast cancer cells for the treatment of breast cancer. In some embodiments, the breast cancer is triple negative breast cancer, estrogen receptor-positive breast cancer, metastatic breast cancer, HER2 positive breast cancer, and variants thereof.

In some embodiments, the cancer or cells of the cancer are resistant to primary cancer therapeutic treatment prior to administering the inhibitor of glycogen metabolism and administering the inhibitor of glycogen metabolism is carried out in an amount effective to re-sensitize the cancer cells to cancer therapy.

In some embodiments, the cancer or cells of the cancer are not resistant to primary cancer therapeutic treatment, and the inhibitor of glycogen metabolism is administered in an amount effective to inhibit, slow, or prevent cancer cell resistance to another cancer therapeutic treatment.

In some embodiments, the inhibitor of glycogen metabolism and additional cancer therapy, when both used, are administered concurrently. In some embodiments, the inhibitor of glycogen metabolism is administered prior to administering the additional cancer therapy. It is contemplated that, in some embodiments, the inhibitor of glycogen metabolism is administered after the additional cancer therapy is administered.

In accordance with the methods described herein, administration of the inhibitor of glycogen metabolism (and optional additional cancer therapy) is carried out by systemic or local administration. Suitable modes of systemic administration of the therapeutic agents and/or combination therapeutics disclosed herein include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes. In certain embodiments, the therapeutic agents of the methods described herein are delivered orally. Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the type of cancer to be treated.

A therapeutically effective amount of the inhibitor of glycogen metabolism alone or in combination with an additional cancer therapy in the methods disclosed herein is an amount that, when administered over a particular time interval, results in achievement of one or more therapeutic benchmarks (e.g., slowing or halting of tumor growth, tumor regression, cessation of symptoms, etc.). The inhibitor of glycogen metabolism alone or in combination with the additional cancer therapy for use in the presently disclosed methods may be administered to a subject one time or multiple times. In those embodiments where the compound is administered multiple times, they may be administered at a set interval, e.g., daily, every other day, weekly, or monthly. Alternatively, they can be administered at an irregular interval, for example on an as-needed basis based on symptoms, patient health, and the like. For example, a therapeutically effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer or the regression of the cancer is monitored during or after the treatment, for example, by a multiparametric ultrasound (mpUS), multiparametric magnetic resonance imaging (mpMRI), and nuclear imaging (positron emission tomography [PET]) of the subject. The dosage of the therapeutic agent(s) or combination therapy administered to the subject can be increased or decreased depending on the status of the cancer or the regression of the cancer detected.

The skilled artisan can readily determine this amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population). Ideally, the therapeutically effective amount does not exceed the maximum tolerated dosage at which 50% or more of treated subjects experience side effects that prevent further drug administrations.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like. In some embodiments, the amount of the inhibitor of glycogen metabolism administered is between about 1.0 mg/kg and about 100 mg/kg. For example, inhibitor of glycogen metabolism may be administered in an amount of about 1.0 mg/kg, about 5.0 mg/kg, about 10.0 mg/kg, about 15.0 mg/kg, about 20.0 mg/kg, about 25.0 mg/kg, about 30.0 mg/kg, about 35.0 mg/kg, about 40.0 mg/kg, about 45.0 mg/kg, about 50.0 mg/kg, about 55.0 mg/kg, about 60.0 mg/kg, about 65.0 mg/kg, about 70.0 mg/kg, about 75.0 mg/kg, about 80.0 mg/kg, about 85.0 mg/kg, about 90.0 mg/kg, about 95.0 mg/kg, and about 100.0 mg/kg.

The effectiveness of the methods of the present application in treating cancer may be evaluated, for example, by assessing changes in tumor burden and/or disease progression following treatment with the inhibitor of glycogen metabolism alone or in combination with the one or more additional cancer therapy as described herein according to the Response Evaluation Criteria in Solid Tumours (Eisenhauer et al., “New Response Evaluation Criteria in Solid Tumours: Revised RECIST Guideline (Version 1.1),” Eur. J. Cancer 45(2):228-247 (2009), which is hereby incorporated by reference in its entirety). In some embodiments, tumor burden and/or disease progression is evaluated using imaging techniques including, e.g., X-ray, computed tomography (CT) scan, magnetic resonance imaging, multiparametric ultrasound (mpUS), multiparametric magnetic resonance imaging (mpMRI), and nuclear imaging (positron emission tomography [PET]) (Eisenhauer et al., “New Response Evaluation Criteria in Solid Tumours: Revised RECIST Guideline (Version 1.1),” Eur. J. Cancer 45(2):228-247 (2009), which is hereby incorporated by reference in its entirety). Cancer regression or progression may be monitored prior to, during, and/or following treatment with one or more of the therapeutic agents described herein.

In some embodiments, the therapeutically effective amount of the inhibitor of glycogen metabolism is the amount that results in a reduction of the effective dose of the additional cancer therapy. In other words, the combination of inhibitor of glycogen metabolism and additional cancer therapeutic may allow for a reduced dosing level of the additional cancer therapy as compared to when that cancer therapy is administered as a monotherapy. In some embodiments, the dose of the additional cancer therapy is reduced by 1%, 2.5%, 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50% when administered in combination with an inhibitor of glycogen metabolism. In some embodiments, the dose of the additional cancer therapy is reduced by more than 50% (e.g., any number between 50-100) when administered in combination with the inhibitor of glycogen metabolism. In some embodiments, administering the inhibitor of glycogen metabolism in combination with the additional cancer therapy lowers the dose of the additional cancer therapy to a dose having reduced toxicity and/or side-effects as compared to the monotherapeutic dose of the primary therapeutic. Thus, in some embodiments, administering the inhibitor of glycogen metabolism in combination with a lower dose of additional cancer therapy relative to a monotherapeutic dose of the primary therapeutic results in a reduction in toxicity to the subject and/or a reduction in primary therapeutic related side effects.

In some embodiments, the response to treatment with the methods described herein results in at least about 1%, 2%, 3%, 4%, 5%, 6%, 7%, 8%, 9%, 10%, 20%, 30%, 40%, 50%, 60%, 70%, 80%, 90%, 95%, 99%, or 100% decrease in tumor size or cancer expression level, as compared to baseline tumor size or cancer cell expression level. Thus, the response to treatment with any of the methods described herein may be partial (e.g., at least a 30% decrease in tumor size, as compared to baseline tumor size) or complete (elimination of the tumor).

In some embodiments, the effectiveness of the methods described herein may be evaluated, for example, by assessing drug induced cancer cell differentiation following treatment with the inhibitor of glycogen metabolism alone or in combination with the one or more additional cancer therapeutics.

In some embodiments, the methods described herein may be effective to inhibit disease progression, inhibit tumor growth, reduce primary tumor size, relieve tumor-related symptoms, inhibit tumor-secreted factors (e.g., tumor-secreted hormones), delay the appearance of primary or secondary cancer tumors, slow development of primary or secondary cancer tumors, decrease the occurrence of primary or secondary cancer tumors, slow or decrease the severity of secondary effects of disease, arrest tumor growth, and/or achieve regression of cancer in a selected subject. Thus, the methods described herein are effective to increase the therapeutic benefit to the selected subject.

In some embodiments, the methods described herein reduce the rate of tumor growth in the selected subject by at least about 1%, 2%, 3%, 4%, 5%, 6%, 7%, 8%, 9%, 10%, 20%, 30%, 40%, 50%, 60%, 70%, 80%, 90%, 95%, 99% (or any number or range between 1-99), or more. In certain embodiments, the methods described herein reduce the rate of tumor invasiveness in the selected subject by at least about 1%, 2%, 3%, 4%, 5%, 6%, 7%, 8%, 9%, 10%, 20%, 30%, 40%, 50%, 60%, 70%, 80%, 90%, 95%, 99% (or any number or range between 1-99), or more. In specific embodiments, the methods described herein reduce the rate of tumor progression in the selected subject by at least about 1%, 2%, 3%, 4%, 5%, 6%, 7%, 8%, 9%, 10%, 20%, 30%, 40%, 50%, 60%, 70%, 80%, 90%, 95%, 99% (or any number or range between 1-99), or more. In various embodiments, the methods described herein reduce the rate of tumor recurrence in the selected subject by at least about 1%, 2%, 3%, 4%, 5%, 6%, 7%, 8%, 9%, 10%, 20%, 30%, 40%, 50%, 60%, 70%, 80%, 90%, 95%, 99% (or any number or range between 1-99), or more. In some embodiments, the methods described herein reduce the rate of metastasis in the selected subject by at least about 1%, 2%, 3%, 4%, 5%, 6%, 7%, 8%, 9%, 10%, 20%, 30%, 40%, 50%, 60%, 70%, 80%, 90%, 95%, 99% (or any number or range between 1-99), or more.

A second aspect of the present disclosure is directed to a method of inhibiting tumor growth in a subject. The method includes administering to a subject having a tumor an inhibitor of glycogen metabolism under conditions effective to treat the tumor, wherein the tumor is characterized by cells having an increased glycogen expression or activity relative to corresponding non-tumor cells of similar origin.

This aspect is carried out in accordance with the previously described aspect.

A third aspect of the present disclosure is directed to a combination therapy. The combination therapy includes an inhibitor of glycogen metabolism, and a cancer therapeutic.

As used herein, the term “combination therapy” refers to the administration of two or more therapeutic agents, i.e., one or more inhibitor of glycogen metabolism in combination with a cancer therapeutic (interchangeably referred to herein as a “primary cancer therapeutic”), suitable for the treatment of cancer, such as a solid malignant tumor. In some embodiments, the combination therapy is co-administered in a substantially simultaneous manner, such as in a single capsule or other delivery vehicle having a fixed ratio of active ingredients. In some embodiment, the combination therapy is administered in multiple capsules or delivery vehicles, each containing an active ingredient. In some embodiments, the therapeutic agents of the combination therapy are administered in a sequential manner, either at approximately the same time or at different times. For example, in some embodiments, the inhibitor of glycogen metabolism is administered as a neo-adjuvant, i.e., it is administered prior to the administration of the cancer therapeutic. In other embodiments, the inhibitor of glycogen metabolism is administered as a standard adjuvant therapy, i.e., it is administered after the administration of the cancer therapeutic. In all of the embodiments, the combination therapy provides beneficial effects of the drug combination in treating cancer, particularly in early stage, aggressive and treatment-resistant cancers as described herein.

In accordance with this and all aspects of the present disclosure, the combination therapy comprises an inhibitor of glycogen metabolism.

In some embodiments, the combination therapy as described herein provides a synergistic effect, as measured by, for example, the extent of the response, the response rate, the time to disease progression, or the survival period, as compared to the effect achievable on dosing with the cancer therapeutic alone at its conventional dose. For example, the effect of the combination treatment is synergistic if a beneficial effect is obtained in a patient that does not respond (or responds poorly) to the cancer therapeutic alone. In addition, the effect of the combination treatment is defined as affording a synergistic effect if the cancer therapeutic is administered at dose lower than its conventional dose and the therapeutic effect, as measured by, for example, the extent of the response, the response rate, the time to disease progression or the survival period, is equivalent to that achievable on dosing conventional amounts of cancer therapeutic. In particular, synergy is deemed to be present if the conventional dose of the cancer therapeutic is reduced without detriment to one or more of the extent of the response, the response rate, the time to disease progression, and survival data, in particular without detriment to the duration of the response, but with fewer and/or less troublesome side-effects than those that occur when conventional doses of each component are used.

In some embodiments, the combination therapeutic encompasses an inhibitor of glycogen metabolism and one or more cancer therapeutic formulated separately, but for administration together. In another embodiment, the combination therapeutic encompasses the inhibitor of glycogen metabolism and one or more cancer therapeutic formulated together in a single formulation. A single formulation refers to a single carrier or vehicle formulated to deliver effective amounts of both therapeutic agents in a unit dose to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension. In yet another embodiment, the vehicle is a nanodelivery vehicle.

In some embodiments, the cancer therapeutic is selected from a MAPK inhibitor, a pentose phosphate pathway inhibitor, an inhibitor of cancer stem cell formation, a cyclin dependent kinase (CDK) inhibitor, a tyrosine kinase inhibitor, an anthracycline antibiotic, a glucose molecule, a pyruvic acid derivative, a phosphogluconate dehydrogenase inhibitor, an activator of Interferon/JAK1/STAT1 signaling, a phosphoinositide 3-kinase (PI3K) inhibitor, a PTEN activator, an anti-estrogen, or a combination thereof. In some embodiments, the cancer therapeutic is a thyroid hormone receptor beta-1 (TRβ) agonist. In some embodiments, the cancer therapeutic is not a thyroid hormone receptor beta-1 (TRβ) agonist.

Suitable nanodelivery vehicles for the delivery of the inhibitor of glycogen metabolism and one or more cancer therapeutics either together or separately, are known in the art and include, for example and without limitation, nanoparticles such as albumin particles (Hawkins et al., “Protein nanoparticles as drug carriers in clinical medicine,” Advanced Drug Delivery Reviews 60(8):876-885 (2008), which is hereby incorporated by reference in its entirety), cationic bovine serum albumin nanoparticles (Han et al., “Cationic bovine serum albumin based self-assembled nanoparticles as siRNA delivery vector for treating lung metastasis cancer,” Small 10(3)(2013), which is hereby incorporated by reference in its entirety), gelatin nanoparticles (Babaei et al., “Fabrication and evaluation of gelatine nanoparticles for delivering of anti-cancer drug,” Int'l J. NanoSci. Nanotech. 4:23-29 (2008), which is hereby incorporated by reference in its entirety), gliadin nanoparticles (Gulfam et al., “Anticancer drug-loaded gliadin nanoparticles induced apoptosis in breast cancer cells,” Langmuir 28:8216-8223 (2012), which is hereby incorporated by reference in its entirety), zein nanoparticles (Aswathy et al., “Biocompatible Fluorescent Zein Nanoparticles for Simultaneous Bioimaging and Drug Delivery Application,” Advances in Natural Sciences: Nanoscience and Nanotechnology 3(2)(2012), which is hereby incorporated by reference in its entirety), and casein nanoparticles (Elzoghby et al., “Ionically-crosslinked Milk Protein Nanoparticles as Flutamide Carriers for Effective Anticancer Activity in Prostate Cancer-bearing Rats,” Eur. J. Pharm. Biopharm. 85(3):444-451 (2013) which is hereby incorporated by reference in its entirety); liposomes (Feldman et al., “First-in-man Study of CPX-351: A Liposomal Carrier Containing Cytarabine and Daunorubicin in a Fixed 5:1 Molar Ratio for the Treatment of Relapsed and Refractory Acute Myeloid Leukemia,” J. Clin. Oncol. 29(8):979-985 (2011); Ong et al., “Development of stealth liposome coencapsulating doxorubicin and fluoxetine,” J. Liposome Res. 21(4):261-271 (2011); and Sawant et al., “Palmitoyl ascorbate-modified liposomes as nanoparticle platform for ascorbate-mediated cytotoxicity and paclitaxel co-delivery,” Eur. J. Pharm. Biopharm. 75(3):321-326 (2010), which are hereby incorporated by reference in their entirety); polymeric nanoparticles, including synthetic polymers, such as poly-ε-caprolactone, polyacrylamine, and polyacrylate, and natural polymers, such as, e.g., albumin, gelatin, or chitosan (Agnihotri et al., “Novel interpenetrating network chitosan-poly(ethylene oxide-g-acrylamide) hydrogel microspheres for the controlled release of capecitabine,” Int. J. Pharm. 324:103-115 (2006); Bilensoy et al., “Intravesical cationic nanoparticles of chitosan and polycaprolactone for the delivery of Mitomycin C to bladder tumor,” Int. J. Pharm. 371:170-176 (2009), which are hereby incorporated by reference); dendrimer nanocarriers (e.g., poly(amido amide) (PAMAM)) (Han et al., “Peptide conjugated PAMAM for targeted doxorubicin delivery to transferrin receptor overexpressed tumors,” Mol. Pharm. 7:2156-2165 (2010); and Singh et al., “Folate and Folate-PEG-PAMAM dendrimers: synthesis, characterization, and targeted anticancer drug delivery potential in tumor bearing mice,” Bioconjugate Chem. 19:2239-2252 (2008), which are hereby incorporated by reference in their entirety); silica nanoparticle (e.g., xerogels and mesoporous silica nanoparticles) (He et al., “A pH-responsive mesoporous silica nanoparticles based multi-drug delivery system for overcoming multidrug resistance,” Biomaterials 32:7711-7720 (2011); Prokopowicz M., “Synthesis and in vitro characterization of freeze-dried doxorubicin-loaded silica xerogels,” J Sol-Gel Sci. Technol. 53:525-533 (2010); Maver et al., “Novel hybrid silica xerogels for stabilization and controlled release of drug,” Int. J. Pharm. 330:164-174 (2007), which are hereby incorporated by reference in their entirety).

The therapeutic agents and combination therapeutics for use in the methods described herein can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington: The Science and Practice of Pharmacy, 20th edition, ed. Alfonso R. Gennaro (2000), which is incorporated herein by reference in its entirety.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Reference to therapeutic agents described herein includes any analog, derivative, isomer, metabolite, pharmaceutically acceptable salt, pharmaceutical product, hydrate, N-oxide, crystal, polymorph, prodrug or any combination thereof. In certain embodiments, the therapeutic agents disclosed herein may be in a prodrug form, meaning that it must undergo some alteration (e.g., oxidation or hydrolysis) to achieve its active form.

The therapeutic agents in a free form can be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds disclosed herein.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

### EXAMPLES

### Example 1—STAR Methods

Immunofluorescent Analysis of Patient-Derived Tissue Microarray-Thyroid tissue microarray slides (US Biomax, Inc, TH641) were baked at 60° C. for 90 minutes and deparaffinized with three changes of xylene (Sigma-Aldrich) and two changes each of sequential alcohol dilutions (100%, 95%, 70%, 50%). After a water rinse, slides were immersed in 1× Dako (Agilent Technologies) for 20 minutes at 95° C. for antigen retrieval. Cooled slides were washed 3× in PBS and blocked for 1 hour at room temperature in 10% normal goat serum (Jackson ImmunoResearch) diluted in 5% BSA in PBS with 0.3% Triton X-100. Primary antibodies, diluted in PBS containing 10% normal goat serum, 5.0% BSA, and 0.3% Triton X-100, were added and incubated overnight at 4° C. Slides were washed 7× with 5.0% BSA in PBS. Secondary antibodies, diluted in 5.0% BSA in PBS, were applied for 1 hour at room temperature. The TMA slides were washed, stained with 10 μg/mL DAPI (Thermo Fisher Scientific) for 15 minutes, and then washed in 5.0% BSA in PBS. Slides were mounted using Dako mounting medium, and proteins were detected using antibodies optimized for immunofluorescence. Images were captured using a Zeiss LSM510 META confocal microscope and quantified by ImageJ.

Periodic Acid Schiff Staining on Patient-Derived Tissue Microarray—After immunofluorescent imaging, TMA slides were rinsed with H₂O and stained for glycogen using the Periodic Acid Schiff (PAS) Stain Pack (Tyr Scientific).

RNA Extraction and Quantitative Real-Time PCR (qRT-PCR)—Total RNA was extracted using RNeasy Plus Kit (Qiagen) according to manufacturer's protocol. cDNA was synthesized using LunaScript® RT SuperMix Kit (New England Biolabs). Gene expression to validate RNA-seq analysis was quantified by qRT-PCR using Luna® Universal qPCR Master Mix (New England Biolabs) on a QuantStudio 3 real-time PCR system (Thermo Fisher Scientific). Fold change in gene expression compared to endogenous controls was calculated using the ddCT method. Primer sequences (Eurofins Genomics) are indicated in Table 4.

Immunoblot Analysis—Immunoblots were conducted as described previously (see, e.g., Davidson et al., “Thyroid Hormone Receptor Beta Inhibits PI3kAakt-mTOR Signaling Axis in Anaplastic Thyroid Cancer Via Genomic Mechanisms,” Journal of the Endocrine Society 5(8):bvab102 (2021); Davidson, C. D., and Carr, F. E., “Review of Pharmacological Inhibition of Thyroid Cancer Metabolism,” Journal of Cancer Metastasis and Treatment 7:45 (2021); and Davidson et al., “Thyroid Hormone Receptor Beta as Tumor Suppressor: Untapped Potential in Treatment and Diagnostics in Solid Tumors,” Cancers (Basel) 13:4254 (2021), all of which are hereby incorporated by reference in their entirety). Briefly, proteins were isolated from whole cells in lysis buffer (20 mM Tris-HCl (pH 8), 137 mM NaCl, 10% glycerol, 1% Triton X-100, and 2 mM EDTA), quantified via Pierce™ BCA Protein Assay Kit (Thermo Fisher Scientific), and 25 μg protein/sample were resolved by polyacrylamide gel electrophoresis and immobilized onto nitrocellulose membranes. Membranes were blocked with 5% w/v BSA in TBS and 0.1% v/v Tween20 (Gold Bio, St Louis, MO, USA) for one hour at room temperature and incubated with primary antibodies overnight. Membranes were washed with TBS-0.1% Tween20 and secondary antibodies were applied for 1 hour at room temperature in 5% w/v BSA in TBS and 0.1% v/v Tween20. Immunoreactive proteins were detected by enhanced chemiluminescence (Thermo Scientific) on a ChemiDoc XRS+ (Bio-Rad Laboratories). Densitometry analysis was performed using ImageJ.

Transmission Electron Microscopy of Glycogen Deposits—Cells were seeded in 10 cm dishes and allowed to adhere overnight. CTM was aspirated and cells were rinsed with PBS before application of RPMI with no glucose, 11.1 mM glucose, or 11.1 mM glucose+50 μM CP-91,149 for 24 hours. Cells were then rinsed with PBS and lifted with trypsin for preparation for TEM as described previously. Thwe et al., “Cell-intrinsic Glycogen Metabolism Supports Early Glycolytic Reprogramming Required for Dendritic Cell Immune Responses,” Cell Metabolism 26:558-567.e555 (2017), which is hereby incorporated by reference in its entirety.

In Vitro Cell Staining for Measuring Drug Efficacy—Thyroid cells (4.0×10³) were seeded in 96 well plates with 100 μl CTM and allowed to adhere overnight. 100 μl of CTM containing 2× concentration of compound was overlayed in each well for 48 hours unless otherwise indicated. Each well then received 100 μl of 10% w/v cold trichloroacetic acid (Fisher) and incubated at 4° C. for 1 hour to fix the cells. Each well was rinsed 3× with DI H₂O and stained with 100 μl 0.057% w/v sulforhodamine B (SRB, Sigma-Aldrich) for 30 minutes at room temperature in the dark. Wells were washed 4× with 300 μl 1% v/v acetic acid (Fisher), and remaining SRB stain was solubilized with 200 μl of 10 mM Tris buffer, pH 10.5 (Fisher). Absorbance was measured at 564 nm with a Synergy 2 Multi-Detection Microplate Reader (Agilent Technologies) and normalized to a vehicle control. Drug synergy was evaluated using the coefficient of drug interaction (CDI) formula: CDI=AB/(A×B)×100, where AB is the percent remaining cells of an indicated combination treatment, A is the average percent remaining cells of agent 1 alone, and B is the average percent remaining cells of agent 2 alone. A CDI of ≤0.7 is considered significantly synergistic; CDI=1 is additive; CDI>1.0 is antagonistic. Hao et al., “Effect of Lumiracoxib on Proliferation and Apoptosis of Human Nonsmall Cell Lung Cancer Cells In vitro,” Chin Med J (Engl) 121:602-607 (2008), which is hereby incorporated by reference in its entirety.

PYGB RNA Interference—For validating successful PYGB knockdown, 4.0×10⁴ cells were plated in 12 well plates containing 0.2% Lipofectamine™ 3000 Transfection Reagent and the indicated concentration of siRNA targeting PYGB or control siRNA in 1 ml CTM. RNA was extracted 72 hours later for RT-qPCR analysis and protein was extracted 96 hours later for immunoblotting. For measuring the effect of PYGB knockdown on cell viability, 1.0×10³ cells were seeded in 96 well plates with 0.15% Lipofectamine™ 3000 Transfection Reagent and the indicated concentration of siRNA targeting PYGB or control siRNA in 200 μl CTM for 7 days before conducting the SRB assay. For measuring the effect of PYGB knockdown on glycogen content, 6.5×10⁵ cells were seeded in 10 cm dishes with 0.2% Lipofectamine™ 3000 Transfection Reagent and the indicated concentration of siRNA targeting PYGB or control siRNA in 10 ml CTM for 4 days preceding cell lysis for glycogen determination.

Determination of CP-91,149 Efficacy on ATC Stem Cells—ATC thyrospheres were grown and counted as described previously using RPMI-1640 supplemented with 20 ng/ml each of epidermal growth factor (EGF) and fibroblastic growth factor (FGF2) (GoldBio). Bolf et al., “Thyroid Hormone Receptor Beta Induces a Tumor Suppressive Program in Anaplastic Thyroid Cancer,” Molecular Cancer Research 18(10):1443-1452 (2020), which is hereby incorporated by reference in its entirety. Thyrospheres per well were counted and imaged using a digital camera (Diagnostics Instruments) connected to a Nikon Eclipse TS100 inverted microscope.

Cell Viability Assay for Determination of Live/Dead Cell Ratios—1.5×10⁴ cells were plated in 12 well tissue culture dishes. After adhering overnight, the cells were treated with an inhibitor or vehicle at the indicated concentrations. Every day after treatment for four days, the media were removed, cells were washed with PBS, lifted with trypsin, and diluted 1:4 into trypan blue. The number of surviving cells was counted with a hemocytometer.

In Vitro Determination of Cell Attachment Potential—8505C cells were treated with or without 50 μM CP-91,149 for 24 hours in T75 flasks. Cells were washed with PBS and lifted with trypsin. 2.5×10⁴ cells were seeded in 200 μl of CTM with or without 50 μM CP-91,149 in 96 well plates for the indicated time points preceding staining with SRB to determine relative number of cells able to attach in a short time frame.

Quantification of Levels of Glycogen, Glucose-1-Phosphate, Glucose-6-Phosphate, Lactate, ATP, Glucose, NADPH, and Glutathione—Briefly, cells were seeded in 10 cm dishes (for measuring glycogen and NADPH), 6 well plates (GIP, G6P, lactate, glucose, and glutathione), or 96 well plates (ATP) and allowed to adhere overnight. CTM was aspirated and cells were rinsed with PBS before application of compounds dissolved in CTM for 24 hours, at which point cells were ~90% confluent. CTM was then aspirated, cells were rinsed with PBS, and lysed according to the respective manufacturer protocol (see Table 4) for metabolite analysis.

Metabolic Flux Analysis—Thyroid cells (5.0×10⁴) were plated in a 96 well Seahorse culture plate with compounds of interest diluted in 200 μl CTM for 24 hours. Cells were washed with PBS and equilibrated for 1 hour at 37° C. in a non-CO₂ incubator with compounds of interest diluted in Seahorse RPMI (supplemented with 11.1 mM glucose, 2 mM glutamine, and 5% serum) before analysis of OCR and ECAR in a Metabolic Flux Analyzer (Seahorse Bioscience, North Billerica, MA 96XP).

Quantification of Reactive Oxygen Species—Thyroid cells (1.0-2.0×10⁴) were seeded in a black 96 well plate in 200 μl CTM and allowed to adhere overnight. Wells were washed with PBS and incubated with or without 25 μM H₂DCFDA for 45 minutes at 37° C. in a non-CO₂ incubator with compounds of interest diluted in phenol-red free RPMI (supplemented with 2 mM glutamine and 10% charcoal-stripped serum). Wells were washed with PBS and received compounds of interest diluted in phenol-red free, supplemented RPMI and incubated at 37° C. in a SpectraMax M4 Microplate Reader (Molecular Devices) to record fluorescent measurements (Ex/Em=485/535 nm) every 15 minutes overnight. Maximum fluorescence intensity was determined (~6 hours), background signal (—H₂DCFDA) was subtracted from each group, and corrected values were normalized to the vehicle control.

Statistical Analysis—All statistical analyses were performed using GraphPad Prism software. Paired comparisons were conducted by unpaired Student's t-test assuming two-tailed distribution. Group comparisons were made by one-way ANOVA followed by Dunnett's or Tukey's multiple comparison test as appropriate. Two-way ANOVA followed by a Tukey's multiple comparison test was conducted for multigroup analysis. Data are represented as mean±standard deviation or standard error of the mean where indicated. Area under the curve (AUC) at the 95th confidence interval followed by appropriate statistical tests was used to evaluate statistical differences in indicated assays. Sample size estimation was done taking into consideration previous experience with animal strains, assay sensitivity, and tissue collection methodology. p<0.05 was considered statistically significant.

### Example 2—Experimental Model and Subject Details

Culture of Thyroid Cell Lines—Unless otherwise stated, cells were cultured in complete thyroid media (CTM): RPMI-1640 growth media with L-glutamine (300 mg/L), sodium pyruvate and nonessential amino acids (1%) (Corning Inc.) supplemented with 10% fetal bovine serum (Peak Serum) and penicillin-streptomycin (200 IU/L) (Corning Inc.) at 37° C., 5% CO₂, and 100% humidity. All data were generated from cell lines within 1-5 passages from acquisition. See Table 4 for detailed information on cell line sources and identifiers. All cell lines were tested regularly for Mycoplasma via PCR and validated via short-tandem repeat profiling prior to use. All cell lines were generated from female patients.

Ethics statement and animal modeling—All animal procedures were approved by Institutional Animal Care and Use Committee (IACUC) at the University of Vermont. All animals were maintained in pathogen-free conditions and cared for in accordance with IACUC policies and certification. All surgeries were performed with isoflurane anesthesia. Temperature-controlled post-surgical monitoring was implemented to minimize suffering. Mice carrying anaplastic thyroid tumors were euthanized at designated time points for tumor collection. Signs of ulceration or a maximum individual tumor size of 2000 mm³ were used as a protocol-enforced endpoint.

In Vivo Evaluation of CP-91,149 and Sorafenib—The xenograft experiment was approved by the Animal Care and Use Committee of the University of Vermont (Protocol X0-018). Four-week-old athymic female nude mice (outbred homozygous nude Foxn1^{nu}/Foxn1^{nu}) were purchased from Jackson Laboratory (Bar Harbor, ME, USA) and were allowed to acclimatize for one week. Mice were given food and water ad libitum. Mice were anesthetized using 3% isoflurane delivered at 1.5 L/min, and tumors were established by subcutaneously injecting 1×10⁶ 8505C cells with a 26G needle in 100 μL of high concentration Matrigel (Corning Inc.) diluted 1:2 with base RPMI-1640 into each flank of 24 mice. One week later, mice were sorted into four treatment groups to achieve approximately equal body weights between mice. Mice were then administered 50 mg/kg CP-91,149, 10 mg/kg sorafenib, both CP-91,149 and sorafenib, or vehicle control every day for 6-7 days a week via intraperitoneal injection using 27G needles. These doses were based on previous reports. Gan et al., “3-hydroxyanthranic Acid Increases the Sensitivity of Hepatocellular Carcinoma to Sorafenib by Decreasing Tumor Cell Stemness,” Cell Death Discov 7:173 (2021); Martin et al., “Discovery of a Human Liver Glycogen Phosphorylase Inhibitor That Lowers Blood Glucose In vivo,” Proc Natl Acad Sci USA 95:1776-1781 (1998); and Wu et al., “Antrodia cinnamomea Boosts the Anti-tumor Activity of Sorafenib in Xenograft Models of Human Hepatocellular Carcinoma,” Sci Rep 8:12914 (2018), all of which are hereby incorporated by reference in their entirety. Pharmacological agents were dissolved in 100% DMSO and diluted daily in 30% DMSO, 40% PEG300, and 30% PBS and incubated at 55° C. for 10 minutes prior to vortexing to encourage complete solubilization. Tumor dimensions were measured with digital calipers, and the volumes were calculated by the following formula: (Π×a×b²)/6, where a represents the largest diameter and b is the perpendicular diameter. The body weight of each animal was taken twice a week to measure toxicity. Mice were euthanized with carbon dioxide, and the tumors were harvested, fixed with formalin (Thermo Fisher Scientific), and stored at 4° C. prior to slide sectioning and immunofluorescence analysis.

TABLE 4
Key Resources

| | | |
|---|---|---|
| Reagent or | | |
| Resource | Source | Identifier |

Antibodies (application & dilution)

| | | |
|---|---|---|
| Anti-GYS1 (WB, | Abcam | Cat# ab40810; RRID: AB_732660 |
| 1:10000 and IF, | | |
| 1:500) | | |
| Anti-pGYS1 (WB, | Millipore Sigma | Cat# 07-817; RRID: AB_568824 |
| 1:1000) | | |
| Anti-PYGL (WB, | Abcam | Cat# ab198268; RRID: AB_2894845 |
| 1:1000 and IF, | | |
| 1:500) | | |
| Anti-PYGB (WB, | Invitrogen | Cat# PA5-28022; RRID: AB_2545498 |
| 1:1500 and IF, | | |
| 1:500) | | |
| Pro and cleaved | Cell Signaling | Cat# 9542; RRID: AB_2160739 |
| PARP (WB, | Technology | |
| 1:1000) | | |
| Pro and cleaved | Cell Signaling | Cat# 9494; RRID: AB_2068141 |
| Caspase 7 (WB, | Technology | |
| 1:500) | | |
| Pro and cleaved | Cell Signaling | Cat# 9502; RRID: AB_2068621 |
| Caspase 9 (WB, | Technology | |
| 1:1000) | | |
| Ki-67 (WB, | Thermo Fisher | Cat# PA5-116479; RRID: AB_2894846 |
| 1:10000 and IF, | Scientific | |
| 1:500) | | |
| GAPDH (WB, | Rockland | Cat# 600-401-A33; RRID: AB_2107593 |
| 1:10000) | Immunochemicals | |
| ß-actin (WB, | Thermo Fisher | Cat# MA5-15739; RRID: AB_10979409 |
| 1:3000) | Scientific | |
| Cleaved PARP (IF | Cell Signaling | Cat# 9544; RRID: AB_2160724 |
| 1:100) | Technology | |
| Horse Anti-Mouse | Cell Signaling | Cat# 7076; RRID: AB_330924 |
| IgG HRP Conjugate | Technology | |
| (WB, 1:10000) | | |
| Goat Anti-Rabbit | Cell Signaling | Cat# 7074; RRID: AB_2099233 |
| IgG HRP Conjugate | Technology | |
| (WB, 1:10000) | | |
| Goat Anti-Rabbit | Cell Signaling | Cat# 4412; RRID: AB_1904025 |
| IgG Alexa Fluor® | Technology | |
| 488 Conjugate (IF, | | |
| 1:500) | | |
| Goat Anti-Mouse | Cell Signaling | Cat# 8890; RRID: AB_2714182 |
| IgG Alexa Fluor® | Technology | |
| 594 Conjugate (IF, | | |
| 1:500) | | |

Media, Supplements, Small Molecule Reagents, and Cell Culture Supplies

| | | |
|---|---|---|
| RPMI (glucose and | Sigma-Aldrich | Cat# R8758 |
| glutamine replete) | | |
| Sodium pyruvate | Corning Inc. | Cat# 25-000-CI |
| Nonessential amino | Corning Inc. | Cat# 25-025-CI |
| acids | | |
| Fetal bovine serum | Peak Serum | Cat# PS-FB2 |
| Charcoal stripped | Sigma-Aldrich | Cat# F6765 |
| serum | | |
| Penicillin- | Corning Inc. | Cat# 30-002-CI |
| Streptomycin | | |
| RPMI (No glucose, | Gibco | Cat# 11879020 |
| No pyruvate, | | |
| glutamine replete) | | |
| RPMI (No glucose, | Gibco | Cat# A2494201 |
| No pyruvate, No | | |
| glutamine, No | | |
| phenol red) | | |
| Agilent Seahorse | Agilent | Cat# 103576-100 |
| XF RPMI | Technologies | |
| Glutamine | Sigma-Aldrich | Cat# G7513 |
| Glucose | Research Products | Cat# G32040 |
| | International | |
| Recombinant | GoldBio | Cat# 1140-02-10 |
| Human Fibroblast | | |
| Growth Factor 2 | | |
| Recombinant | GoldBio | Cat# 1150-04-100 |
| Human Epidermal | | |
| Growth Factor | | |
| DMSO | Sigma-Aldrich | Cat# D2650 |
| Trypsin-EDTA | Gibco | Cat# 25200056 |
| PBS | Corning, Inc. | Cat# 21-040-CV |
| Trypan blue | Corning, Inc. | Cat# 25-900-CI |
| Buparlisib | MedChemExpress | Cat# Hy70063 |
| Alpelisib | MedChemExpress | Cat# Hy-15244 |
| Sorafenib | MedChemExpress | Cat# Hy-10201 |
| Lapatinib | MedChemExpress | Cat# Hy-50898 |
| Palbociclib | MedChemExpress | Cat# Hy50767 |
| Lenvatinib | MedChemExpress | Cat# HY-10981 |
| Doxorubicin | Abcam | Cat# ab120629 |
| CP-91,149 | Selleckchem | Cat# S2717 |
| CP 316819 | Tocris Bioscience | Cat# 3542 |
| 6-AN | Cayman Chem | Cat# 10009315 |
| 2-DG | Selleckchem | Cat# S4701 |
| 3-BP | Cayman Chem | Cat# 19068 |
| Guaiacol | Acros Organics | Cat# AC120192500 |
| yGsy2p-IN-1 | MedChemExpress | Cat# HY-131062 |
| MnTMPyP | Sigma-Aldrich | Cat# 475872 |
| N-Acetylcysteine | MedChemExpress | Cat# HY-B0215 |
| H2DCFDA | Selleckchem | Cat# S9687 |
| NADPH | Santa Cruz | Cat# SC-202725 |
| | Biotechnology | |
| 30% H₂O₂ | Sigma-Aldrich | Cat# 216763 |
| High concentration | Corning, Inc. | Cat# 354248 |
| basement | | |
| membrane matrix | | |
| Matrigel ® | | |
| Polyethylene glycol | Sigma-Aldrich | Cat# 202371 |
| (PEG) 300 | | |

Critical Commercial Assays

| | | |
|---|---|---|
| RNeasy Mini Kit | Qiagen | Cat# 74106 |
| (RNA extraction) | | |
| LunaScript ® RT | New England | Cat# E3010 |
| SuperMix Kit | Biolabs | |
| Luna ® Universal | New England | Cat# M3003 |
| qPCR Master Mix | Biolabs | |
| Lipofectamine ™ | Thermo Fisher | Cat# L3000015 |
| 3000 Transfection | Scientific | |
| Reagent | | |
| Silencer Select | Invitrogen | Cat# 4390844 |
| Negative Control | | |
| siRNA | | |
| Silencer ® Select | Thermo Fisher | Assay ID: s11641 |
| siRNA for PYGB | Scientific | |
| Glycogen | BioVision Inc. | Cat# K646-100 |
| Colorimetric/Fluoro | | |
| metric Assay Kit | | |
| Pierce ™ BCA | Thermo Fisher | Cat# 23225 |
| Protein Assay Kit | Scientific | |
| Glucose-1- | Bio Vision Inc. | Cat# K697 |
| Phosphate | | |
| Colorimetric Assay | | |
| Kit | | |
| EnzyChrom ™ | BioAssay Systems | Cat# EGTT-100 |
| GSH/GSSG Assay | | |
| Kit | | |
| EnzyChrom ™ L- | BioAssay Systems | Cat# ECLC-100 |
| Lactate Assay Kit | | |
| Glucose Assay Kit I | Eton Bioscience | Cat# 1200031002 |
| CellTiter-Glo ® 2.0 | Promega | Cat# G9241 |
| Cell Viability ATP | | |
| Assay | | |
| NADPH Assay Kit | Abcam | Cat# ab186031 |

Staining Reagents

| | | |
|---|---|---|
| Periodic Acid | Tyr Scientific | Cat# TS9-500PK |
| Schiff (PAS) Stain | | |
| Pack | | |
| Trichloroacetic acid | Fisher Bioreagents | Cat# BP555-250 |
| Sulforhodamine B | Sigma-Aldrich | Cat# S1402 |
| Glacial acetic acid | Fisher Bioreagents | Cat# BP2401-500 |
| Tris base | Fisher Bioreagents | Cat# BP152-1 |
| Formal-Fixx ™ | Thermo Fisher | Cat# 9990244 |
| formalin | Scientific | |
| Xylene | Sigma-Aldrich | Cat# 214736 |
| Ethyl alcohol, 200 | Pharmco | Cat# 111000200 |
| proof | | |
| Dako Antigen | Agilent | Cat# S169984-2 |
| Retrieval, pH 6.1 | Technologies | |
| Normal goat serum | Jackson | Cat# 005-000-121, RRID: AB_2336990 |
| | ImmunoResearch | |
| DAPI | Thermo Fisher | Cat# D1306 |
| | Scientific | |
| Dako Fluorescence | Agilent | Cat# S3023 |
| Mounting Medium | Technologies | |

Cell Lines

| | | |
|---|---|---|
| Nthy-ori 3-1 | Sigma-Aldrich | Cat# 90011609, RRID:CVCL 2659 |
| TPC-1 | Mayo Clinic, | Marlow, et al., J Clin Endocrinol Metab, 2018. |
| | Jacksonville FL, | |
| | USA | |
| B-CPAP, FTC-133, | University of | Landa, et al., Clin Cancer Res, 2019. |
| CUTC61, OCUT-2, | Colorado, Denver | |
| 8505C | CO, USA | |

Mouse Model

| 4 wk female J:NU | Jackson Laboratory | Cat# 007850, RRID:IMSR JAX:007850 | |
|---|---|---|---|
| FoxnInu/Foxn Inu | | | |
| Software | | | |
| ImageJ | Open | https://imagej.nih.gov/ij/ | |
| | Source/National | | |
| | Institutes of Health | | |
| GraphPad Prism | GraphPad | https://www.graphpad.com/scientific- | |
| | Software, Inc. | software/prism/ | |
| Image Studio™ | LI-COR | https://www.licor.com/bio/image-studio- | |
| | | lite/download | |
| NIS-Elements | Nikon Instruments | https://www.microscope.healthcare.nikon.com/pr | |
| | | oducts/software/nis-elements | |
| Gen5 Data Analysis | BioTek | https://www.biotek.com/products/software- | |
| | | robotics-software/gen5-microplate-reader-and- | |
| | | imager-software/ | |
| Seahorse Wave | Agilent | https://www.agilent.com/en/product/cell- | |
| Software | Technologies | analysis/real-time-cell-metabolic-analysis/xf- | |
| | | software/seahorse-wave-desktop-software-740897 | |
| | | | Amplicon |
| | | | size |
| Target | Forward (5′-3′) | Reverse (5′-3′) | (bp) |
| GYS1 | ACAACCTGGAGAACT | ATCTGGGACACAGTAG | 118 |
| | TCAAC (SEQ ID NO: 3) | TGAA (SEQ ID NO: 9) | |
| PYGL | TGCCCGGCTACATGA | TGTCATTGGGATAGAG | 162 |
| | ATAACA (SEQ ID NO: 4) | GACCC (SEQ ID NO: 10) | |
| PYGB | ACGCAGCAGCACTAC | TCGCAGGCATTCTGAA | 119 |
| | TAC (SEQ ID NO: 5) | GG (SEQ ID NO: 11) | |
| SLC2A1 | TGGCATCAACGCTGT | AGCCAATGGTGGCATA | 83 |
| | CTTCT (SEQ ID NO: 6) | CACA (SEQ ID NO: 12) | |
| SLC2A3 | CTGAGGACGTGGAG | AATATCAGAGCTGGGG | 154 |
| | AAAACTTG (SEQ ID NO: 7) | TGACCTTC (SEQ ID NO: 13) | |
| SLC2A4 | CAGTGGCTTGGAAGG | CAGGTGAGTGGGAGC | 195 |
| | AAAAGG (SEQ ID NO: 8) | AATCT (SEQ ID NO: 14) | |

### Example 3-Glycogen Phosphorylase Brain Isoform Overexpression Drives Glycogen Breakdown in Thyroid Adenoma and Thyroid Cancers

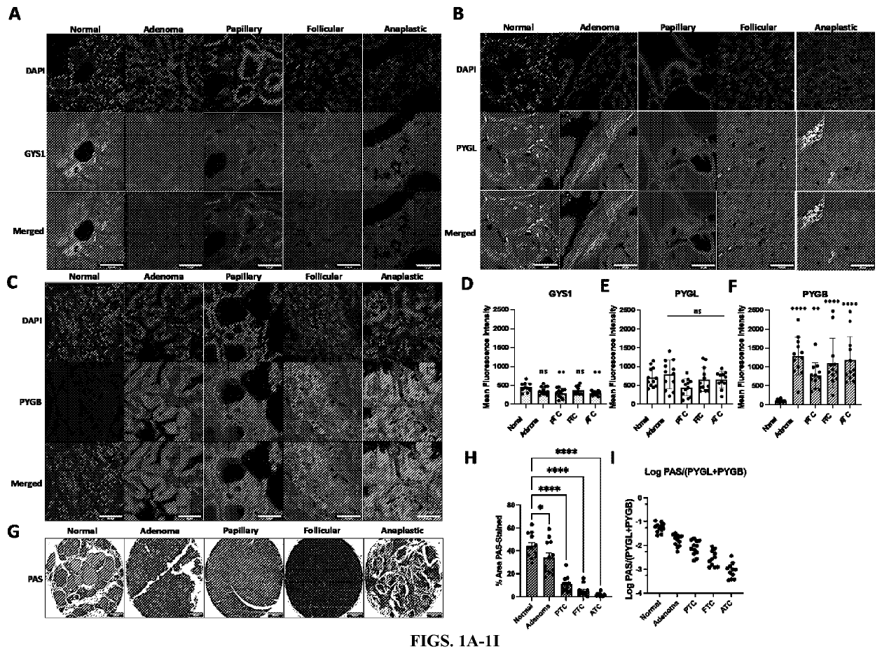

First biopsy samples were profiled from normal thyroid and thyroid adenoma, PTC, FTC, and ATC to measure GYS1, PYGL, and PYGB expression. GYS1 expression was remarkably similar across the spectrum of thyroid cancers. GYS1 was slightly diminished in PTC and ATC compared to normal (FIGS. 1A and 1D). PYGL expression did not change across the spectrum of thyroid differentiation (FIGS. 1B and 1E). Surprisingly, PYGB was virtually undetectable in normal thyroid tissue, and was significantly upregulated in thyroid adenoma and all three forms of thyroid cancer (FIGS. 1C and 1F). Each type of thyroid tissue likely had the potential to metabolize glycogen since all thyroid samples expressed GYS1 and at least one isoform of glycogen phosphorylase. Glycogen content was measured using periodic acid-Schiff staining on the biopsy cores. As expected, glycogen content was inversely related to the presence of PYGB; normal thyroid tissue contained the most glycogen, followed by adenoma, PTC, FTC, and ATC FIGS. 1G-H), and a logarithmic relationship between glycogen staining and the sum of PYGL and PYGB expression was observed (FIG. 1I). Collectively, these results demonstrate that thyroid tissue, normal and malignant, across the spectrum of differentiation express enzymes necessary to store and break down glycogen.

### Example 4-Representative Cell Lines of Normal Thyroid and Thyroid Cancer Cells Metabolize Glycogen Through Expression of Glycogen Synthase and Glycogen Phosphorylase Isozymes

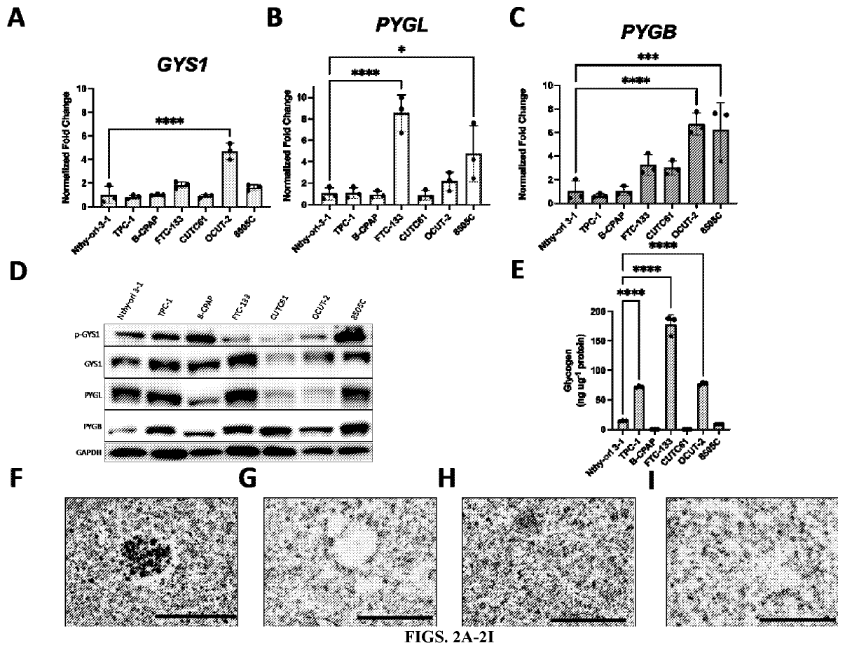

Next, thyroid cell lines were tested to directly investigate the presence and role of glycogen in normal thyroid and thyroid cancer. Cell lines were used representing normal thyroid, PTC, FTC, and ATC from diverse genetic backgrounds. Landa et al., “Comprehensive Genetic Characterization of Human Thyroid Cancer Cell Lines: A Validated Panel for Preclinical Studies,” Clinical Cancer Research 25(10):3141-3151 (2019), which is hereby incorporated by reference in its entirety. On the RNA level, GYS1 expression was comparable across all cell lines except for the OCUT-2 cell line that expressed nearly four times more GYS1 transcript than the normal thyroid cells, Nthy-ori-3-1 (FIG. 2A). This was not unexpected, as GYS1 overexpression has been reported in many cancer cell models. Falantes et al., “Overexpression of gys1, mif, and myc is associated with adverse outcome and poor response to azacitidine in myelodysplastic syndromes and acute myeloid leukemia. Clin Lymphoma Myeloma Leuk 15, 236-244 (2015) and Giatromanolaki et al., “Expression of Enzymes Related to Glucose Metabolism in Non-small Cell Lung Cancer and Prognosis,” Exp Lung Res 43:167-174 (2017), both of which are hereby incorporated by reference in their entirety. Additionally, PYGL expression was comparable in the different types of thyroid cancer cells except the FTC-133 and 8505C cells expressed significantly higher levels (FIG. 2B). In agreement with the biopsy results, PYGB expression correlated with thyroid dedifferentiation; the highest PYGB expression was observed in the ATC cell lines OCUT-2 and 8505C (FIG. 2C). Immunoblot analysis of glycogen metabolism enzymes (FIG. 2D) revealed differences in levels of phosphorylated (deactivated) GYS1. Interestingly, normal, PTC, and an ATC cell line displayed heavily phosphorylated GYS1, whereas both FTC cell lines and the ATC cell line, OCUT-2, displayed decreased GYS1 phosphorylation. The complicated profile is likely reflective of the genetic background of each cell line, as FTC-133, CUTC61, and OCUT-2 all have mutations in the PI3K-Akt-GSK3β pathway, which results in reduced GYS1 phosphorylation. Iida et al., “Hypoxia Promotes Glycogen Synthesis and Accumulation in Human Ovarian Clear Cell Carcinoma,” Int J Oncol 40:2122-2130 (2012), which is hereby incorporated by reference in its entirety. Overall, the thyroid cell line profiles are consistent with the patient tissue staining; PYGB is overexpressed in thyroid cancer. The dynamic expression and activation of glycogen enzymes may impact the differential levels of glycogen in the cell lines, as TPC-1, FTC-133, and OCUT-2 had the highest levels of glycogen (FIG. 2E). Detection of glycogen was validated in the Nthy-ori-3-1 and 8505C cells using tannic acid staining and transmission electron microscopy (TEM). Large (~45 nm), organized glycogen particles were observed in the normal thyroid cells (FIG. 2F) compared to the smaller (~25 nm), dispersed glycogen particles in the 8505C cells (FIG. 2H). Glycogen became nearly undetectable following overnight glucose starvation in both cell lines (FIGS. 2G and 2I), insuring staining specificity.

### Example 5—Glycogen Phosphorylase Inhibition Increases Glycogen Content to Limit Cell Viability and Proliferation and Induce Apoptosis in ATC Cells

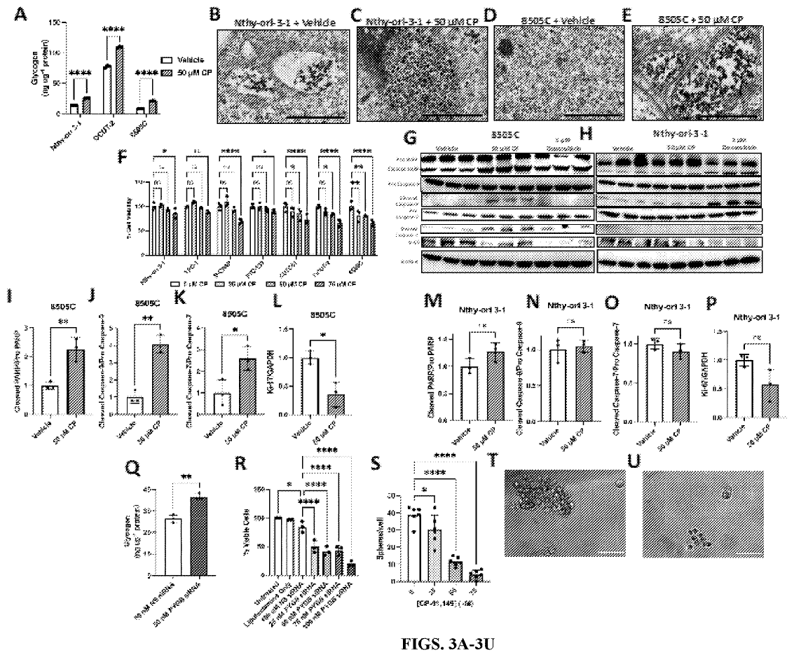

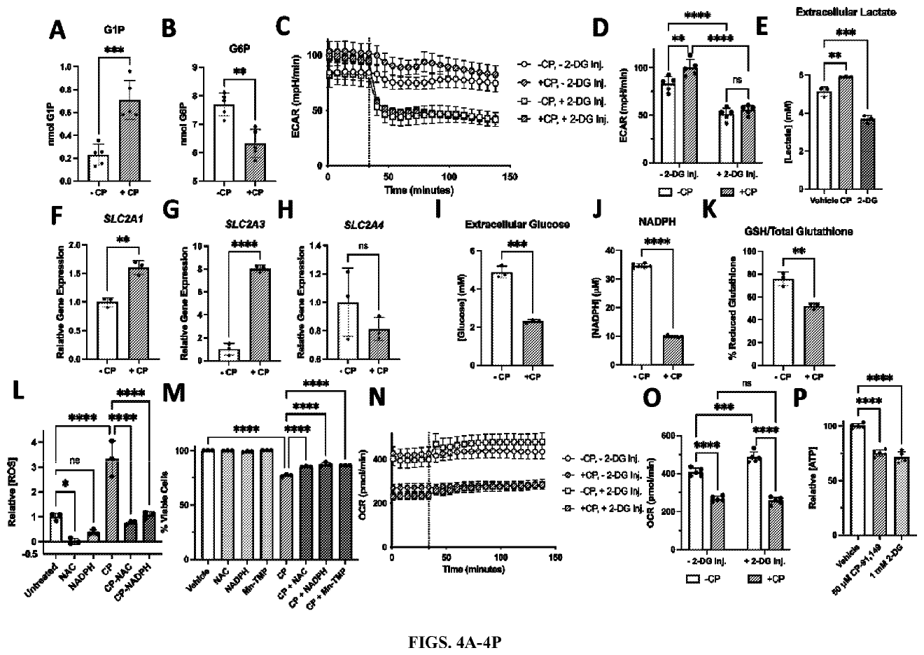

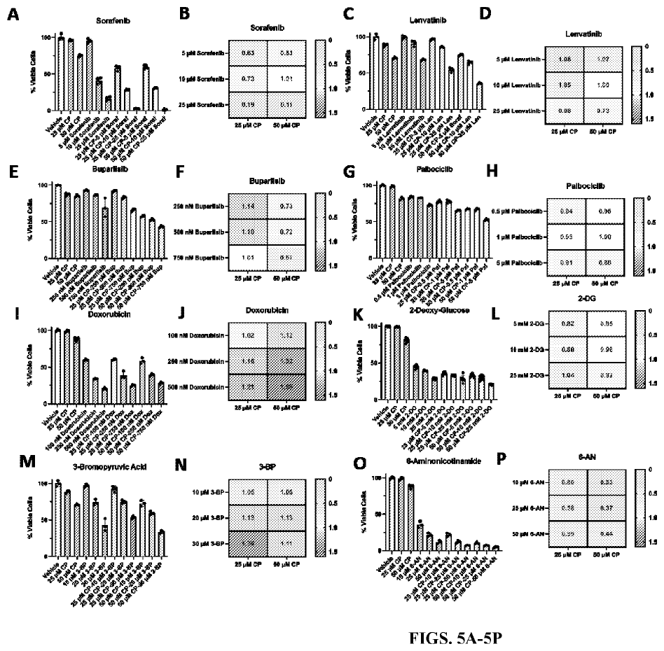

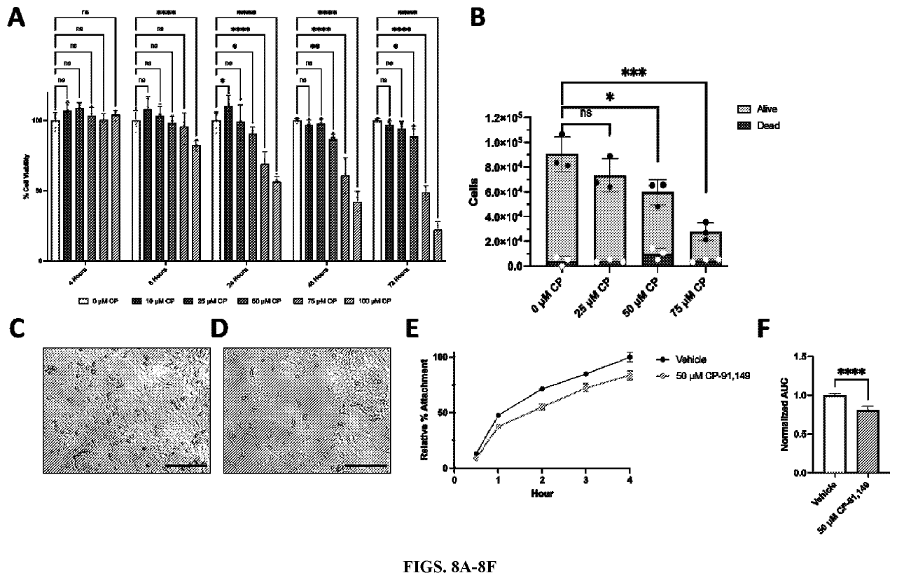

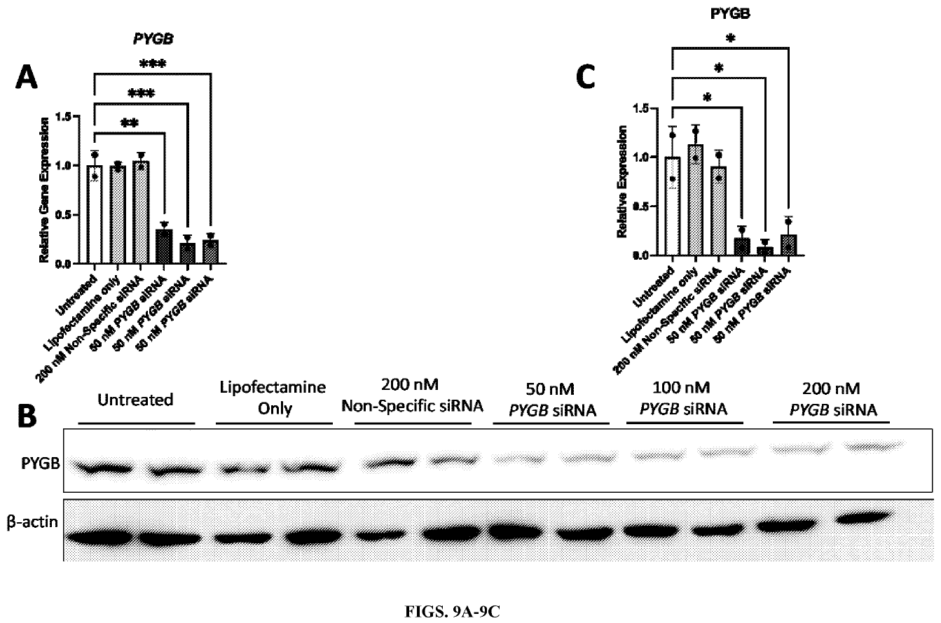

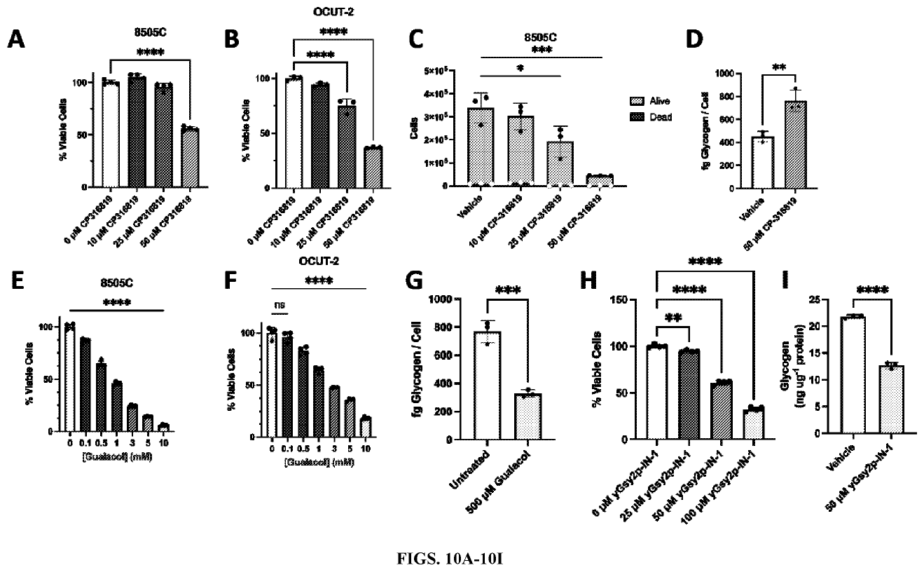

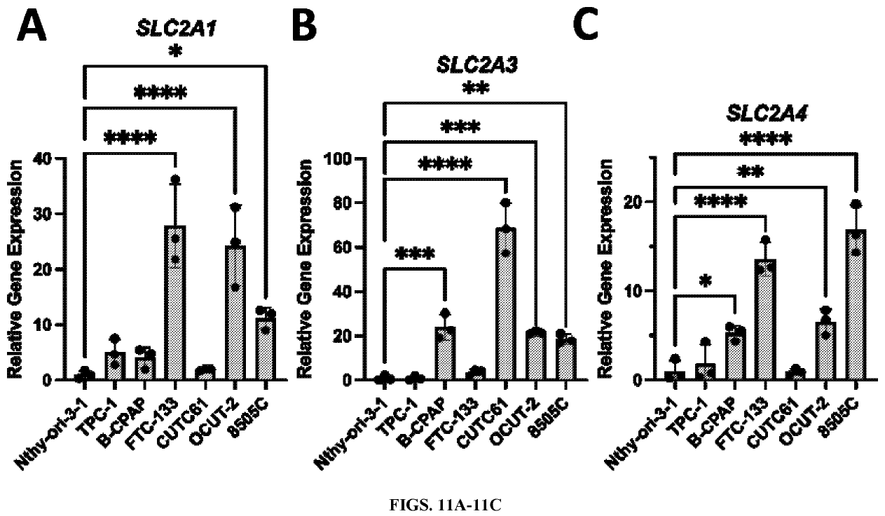

Following the observations that thyroid cancer cells store glycogen and overexpress PYGB, it was reasoned that glycogen phosphorylase could be a promising drug target, particularly in ATC cells for which there is an urgent need for effective, long-term therapeutics. Focus was on the 8505C cells, which are well representative of ATC cells, having no mutations in PIK3CA and low levels of glycogen (FIGS. 1G-1H). Yoo et al., “Recent Improvements in Genomic and Transcriptomic Understanding of Anaplastic and Poorly Differentiated Thyroid Cancers,” Endocrinol Metab 35:44-54 (2020), which is hereby incorporated by reference in its entirety. CP-91,149 (CP) was originally designed to treat diabetes and is a pan glycogen phosphorylase inhibitor by stabilizing the enzyme homodimer in an inactive form. Hayes et al., “Natural Products and Their Derivatives as Inhibitors of Glycogen Phosphorylase: Potential Treatment for Type 2 Diabetes,” Phytochemistry Reviews 13:471-498 (2014) and Martin et al., “Discovery of a Human Liver Glycogen Phosphorylase Inhibitor That Lowers Blood Glucose In vivo,” Proc Natl Acad Sci USA 95:1776-1781 (1998), both of which are hereby incorporated by reference in their entirety. CP significantly increased glycogen content in normal and ATC cells as shown through colorimetry and TEM (FIGS. 3A-3E). CP also modestly but significantly reduced total cell viability in 48 hours at 50 μM in the ATC cells but not the normal thyroid cells (FIGS. 3F and 8A-8D). Inhibiting PYG with CP has been shown to induce intrinsic apoptosis in hepatocellular carcinoma (Barot et al., “Inhibition of Glycogen Catabolism induces Intrinsic Apoptosis and Augments Multikinase Inhibitors in Hepatocellular Carcinoma Cells,” Experimental Cell Research 381(2):288-300 (2019), which is hereby incorporated by reference in its entirety), investigation of the mechanism of cell death in thyroid cells was prompted. CP caused significant PARP, caspase 9, and caspase 7 cleavage in 8505C cells in only six hours (FIGS. 3G and 3I-3K). Additionally, incubation with CP resulted in reduced expression of the proliferation marker Ki-67 (FIGS. 3G and 3L). Conversely, CP did not significantly induce apoptosis in Nthy-ori-3-1 cells as observed by lack of PARP and caspase cleavage (FIGS. 3H and 3M-3O). Proliferation was also not affected in Nthy-ori-3-1 cells as evident from Ki-67 levels (FIGS. 3H and 3P), suggesting that ATC cells are more sensitive to glycogen inhibition than normal thyroid cells. Furthermore, 24 hours of CP treatment inhibited the ability of 8505C cells to attach to fibronectin-coated plates (FIGS. 8E-8F). To validate the specificity of CP-91,149, siRNA targeting PYGB was employed in 8505C cells (FIGS. 9A-9C). A 50% increase in glycogen following PYGB knockdown was observed, as well as a significant decrease in cell viability (FIGS. 3Q-3R). CP efficacy was also assessed on ATC stem cells, which is an important consideration as many inhibitors often fail to kill the pluripotent stem cell population. Gianì et al., “The Possible Role of Cancer Stem Cells in the Resistance to Kinase Inhibitors of Advanced Thyroid Cancer,” Cancers (Basel) 12:2249 (2020) and Zheng et al., “Doxorubicin Fails to Eradicate Cancer Stem Cells Derived From Anaplastic Thyroid Carcinoma Cells: Characterization of Resistant Cells,” Int J Oncol 37:307-315 (2010), both of which are hereby incorporated by reference in their entirety. Excitingly, 8505C stem cells exhibited a concentration-dependent response to CP (FIGS. 3T-3U). This observation may be reflective of stem cells' higher energy requirement and dependence on glycogen as seen in undifferentiated human embryonic stem cells and mesenchymal stem cells. Chen et al., “Synergistic Anti-proliferative Effect of Metformin and Sorafenib on Growth of Anaplastic Thyroid Cancer Cells and Their Stem Cells,” Oncology Reports 33:1994-2000 (2015) and Zhu et al., “Inducible Metabolic Adaptation Promotes Mesenchymal Stem Cell Therapy for Ischemia: A Hypoxia-induced and Glycogen-based Energy Prestorage Strategy,” Arterioscler Thromb Vasc Biol 34:870-876 (2014), both of which are hereby incorporated by reference in their entirety. In addition to PYG inhibition with CP-91,149, ATC cells were sensitive to CP316819. CP316819, like CP-91,149, is an indole-ring containing small molecule inhibitor of PYG that enhanced glycogen storage in rat brains (Suh et al., “Astrocyte Glycogen Sustains Neuronal Activity During Hypoglycemia: Studies With the Glycogen Phosphorylase Inhibitor cp-316,819 ([r-r*,s*]-5-chloro-n-[2-hydroxy-3-(methoxymethylamino)-3-oxo-1-(phenylmethyl) propyl]-1 h-indole-2-carboxamide),” J Pharmacol Exp Ther 321:45-50 (2007), which is hereby incorporated by reference in its entirety). CP316819 limited cell viability and increased the glycogen content of 8505C cells (FIGS. 10A-10D).

### Example 6—ATC Cells are Sensitive to Glycogen Synthase Inhibition

Following the recent success of using naturally occurring inhibitors of GYS1 for treating adult polyglucosan body disease (Kakhlon et al., “Guaiacol as a Drug Candidate for Treating Adult Polyglucosan Body Disease,” JCI Insight 3: e99694 (2018), which is hereby incorporated by reference in its entirety), 8505C and OCUT-2 cells were treated with the flavoring agent guaiacol to decrease ATC cell viability and glycogen levels (FIGS. 10E-10G). Also used was the recently characterized molecule yGsy2p-IN-1, the first specific GYS1 inhibitor (Tang et al., “Discovery and Development of Small-molecule Inhibitors of Glycogen Synthase,” J Med Chem 63:3538-3551 (2020), which is hereby incorporated by reference in its entirety), to limit ATC viability and decrease glycogen content (FIGS. 10H-10I). These data may be the first report of using guaiacol or yGsy2p-IN-1 in cancer cell models to limit cell viability by blocking glycogen utilization, highlighting the potential of targeting glycogen metabolism in aggressive cancers.

### Example 7—CP-91,149 Triggers Glucose Flux in ATC Cells to Fuel Glycolysis but Inhibits NADPH Production, Increases Levels of Reactive Oxygen Species, and Limits Oxidative Phosphorylation

Glycogen provides efficient access to glucose for the cancer cell to use in diverse cellular processes. It was first confirmed that CP resulted in a buildup of the glycogen monomer, glucose-1-phosphate (G1P) and a decrease in the glycolytic intermediate, glucose-6-phosphate (G6P) in 8505C cells (FIGS. 4A-4B). A characteristic of high glycolytic activity is lactate production, which acidifies the extracellular environment. While 2-DG significantly inhibited the extracellular acidification rate (ECAR), it was surprising to find that CP modestly but significantly enhanced ECAR in 8505C cells (FIGS. 4C-4D). These results were confirmed with an extracellular lactate assay (FIG. 4E). it was reasoned that this paradox could be explained by a “glucose stress response” in the cell, in which glucose intake was increased to compensate for glycogen catabolism inhibition. It was chosen to measure expression of the glucose transporters most implicated in ATC; GLUT1, GLUT3, and GLUT4 (FIGS. 11A-11C). Coelho et al., “Metabolic Reprogramming in Thyroid Carcinoma,” Frontiers in Oncology 8:82 (2018); Haber et al., “Glut1 Glucose Transporter Expression in Benign and Malignant Thyroid Nodules,” Thyroid 7:363-367 (1997); and Matsuzu et al., “Differential Expression of Glucose Transporters in Normal and Pathologic Thyroid Tissue,” Thyroid 14:806-812 (2004), all of which are hereby incorporated by reference in their entirety. There was an increase in GLUT1 and GLUT3 (SLC2A1 and SLC2A3) expression following CP treatment, while the glucose-independent GLUT4 (SLC2A4) expression did not change (FIGS. 4F-4H). There was a significant reduction in extracellular glucose following CP treatment as well (FIG. 4I). Unexpectedly, the ECAR readout with 2-DG treatment was equivalent regardless of CP pretreatment (FIGS. 4C-4D). It was previously shown that free glucose and glycogen stores made distinct contributions to ECAR in murine dendritic cells, which suggests that glycogen may not contribute to the glycolytic glucose pool in ATC cells. Thwe et al., “Cell-intrinsic Glycogen Metabolism Supports Early Glycolytic Reprogramming Required for Dendritic Cell Immune Responses,” Cell Metabolism 26:558-567.e555 (2017), which is hereby incorporated by reference in its entirety. Therefore, function of the pentose phosphate pathway (PPP) was investigated following glycogen phosphorylase inhibition. The PPP siphons G6P from glycolysis or glycogenolysis to reduce NAD+ to NADPH via glucose-6-phosphate dehydrogenase (G6PDH). NADPH is a crucial redox metabolite in cancer biology for combating reactive oxygen species (ROS) produced in metabolism and cell signaling. Patra, K. C., and Hay, N., “The Pentose Phosphate Pathway and Cancer,” Trends Biochem Sci 39:347-354 (2014), which is hereby incorporated by reference in its entirety. There was a drastic decrease in NADPH in cells treated with CP compared to control cells (FIG. 4J), which has important implications for the redox potential. Since proteins and nucleotides oxidized by ROS require proteins such as glutathione for repair, reduced and total glutathione was measured following CP treatment and observed a concomitant loss in reduced glutathione (GSH) (FIG. 4K). A three-fold increase was measured in ROS in ATC cells following CP treatment, which was ablated with exogenous NADPH or the glutathione precursor N-acetylcysteine (NAC) (FIG. 4L). To confirm the mechanism of CP-mediated loss in cell viability, CP-treated 8505C cells with NADPH was partially rescued, NAC, and a manganese superoxide mutase mimetic (Mn-TMP) (FIG. 4M). Since high levels of ROS can impair mitochondrial activity and induce apoptosis (Berghella, L., and Ferraro, E., “Early Decrease in Respiration and Uncoupling Event Independent of Cytochrome c Release in pc12 Cells Undergoing Apoptosis,” Int J Cell Biol 2012:643929 (2012); Dranka et al., “Assessing Bioenergetic Function in Response to Oxidative Stress by Metabolic Profiling,” Free Radic Biol Med 51:1621-1635 (2011); and Ricci et al., “Caspase-mediated Loss of Mitochondrial Function and Generation of Reactive Oxygen Species During Apoptosis,” The Journal of Cell Biology 160:65-75 (2003), all of which are hereby incorporated by reference in their entirety), the oxygen consumption rate (OCR) was measured following CP treatment. As expected, CP significantly limited the OCR in ATC cells (FIG. 4O). Intriguingly, vehicle-treated ATC cells could compensate for inhibition of glycolysis with 2-DG by increasing OCR, but the CP-treated cells were unable to increase their oxidative phosphorylation (FIG. 4P).

### Example 8—Glucose and Pyruvate Availability Selectively Modulate ATC Cell Susceptibility to Glycolytic and Glycogenolytic Inhibition

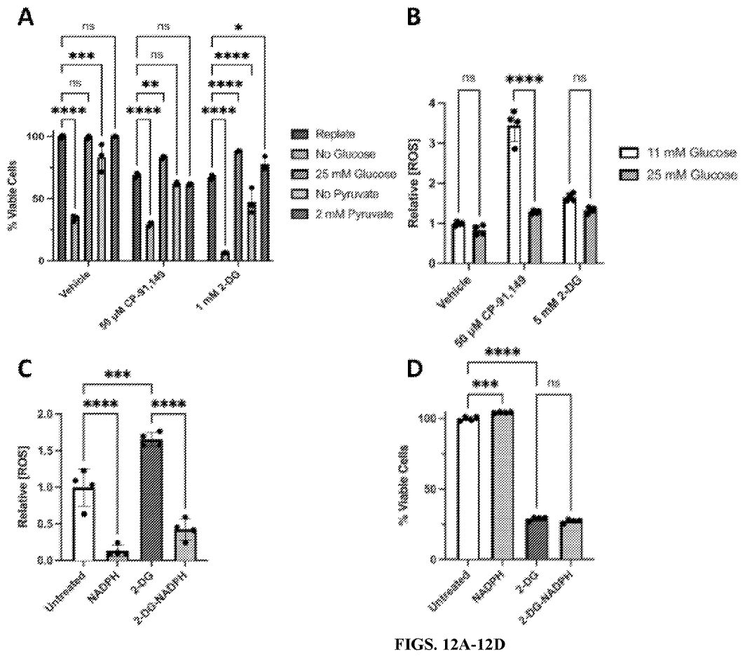

Expanding on the potential for separate glucose pools and functions, levels of glucose and pyruvate were modulated in combination with CP and 2-DG treatment. Since both CP and 2-DG limit the amount of G6P to enter glycolysis or the PPP, it was expected that depriving cells of glucose would decrease cell viability in both treatment groups (FIG. 12A). Excess (25 mM) glucose rescued cell viability after treatment with CP or 2-DG, suggesting that exogenous glucose can compensate loss of both glycolysis and glycogenolysis activity. However, ROS levels were higher in cells treated with CP compared to 2-DG, and exogenous glucose only rescued ROS levels induced by CP (FIG. 12B). Unlike CP, 2-DG only modestly increased ROS levels, and while excess glucose could rescue the viability of cells treated with 2-DG or CP, excess glucose reduced ROS levels only in CP-treated cells. Conversely, supplementing the PPP with exogenous NADPH reduced ROS in both treatment groups (FIGS. 4L and 12C) but only rescued cell viability in the CP-treated cells (FIGS. 4M and 12D). Taken together, these findings suggest that exogenous glucose restores functionality to glycolysis, not the PPP in 2-DG treated cells, and exogenous NADPH restores functionality to the PP in both treatment groups but is only sufficient to rescue viability in CP-treated cells. Pyruvate depletion only significantly impacted the 2-DG-treated cells, suggesting that the cells treated with 2-DG were more dependent on the high energy pyruvate, while the CP-treated cells were able to compensate for loss of pyruvate (FIG. 12A). Likewise, only the 2-DG-treated cells could be rescued with excess (2 mM) pyruvate, whereas the CP-treated cell viability level was nearly identical to the pyruvate-depleted group. These findings support a differential mechanism of CP on cell viability compared to 2-DG.

### Example 9—CP-91,149 Exhibits Drug Synergy with Inhibitors of MAPK Signaling and the Pentose Phosphate Pathway

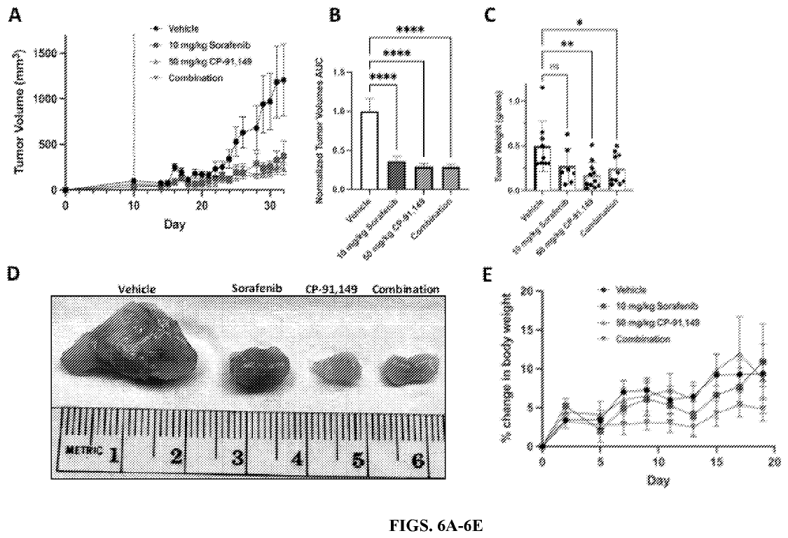

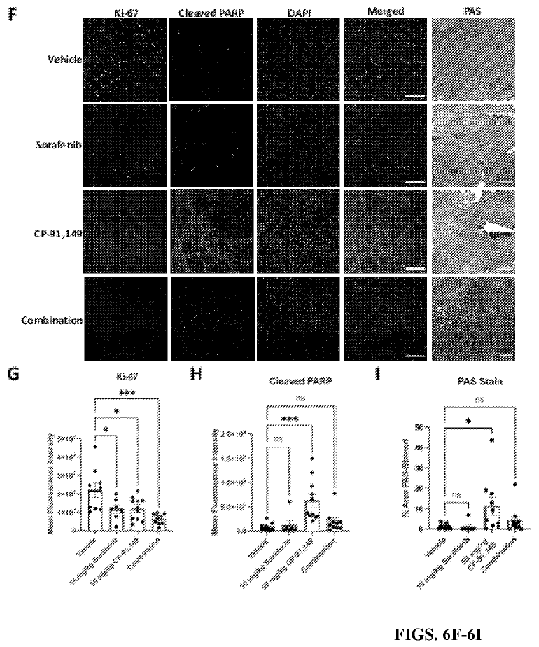

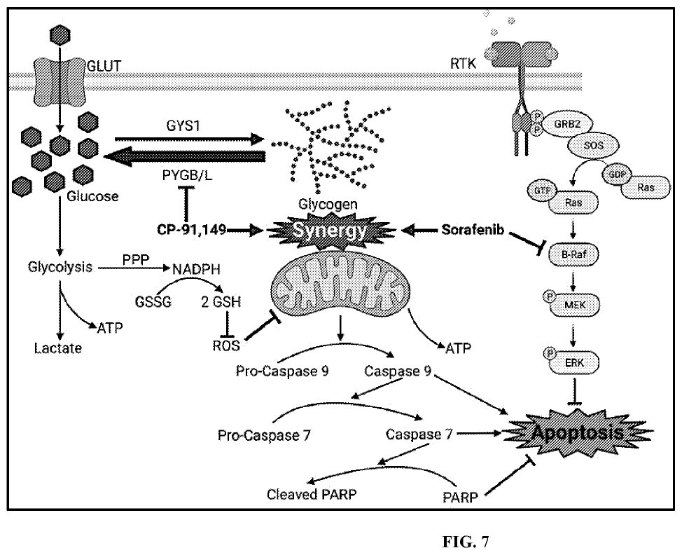

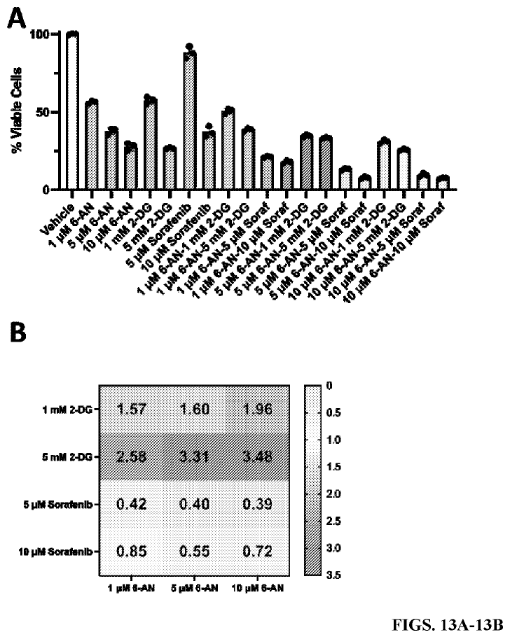

Following the promising in vitro applications of CP in ATC cells, the potential for CP to improve the efficacy of other inhibitors was evaluated by performing cell viability assays to calculate coefficient of drug interaction (CDI) scores to assess drug synergy, additivity, and antagonism. Excitingly, CP displayed a high degree of drug synergy with the multi kinase inhibitor sorafenib (FIGS. 5A-5B). Since sorafenib resistance often develops in ATC tumors, combining the kinase inhibitor with a metabolic inhibitor such as CP could yield more promising results, possibly at lower doses. Interestingly, CP displayed drug additivity rather than synergy with the multi kinase inhibitor lenvatinib (FIGS. 5C-5D), which is approved for aggressive differentiated thyroid cancer (Hao, Z., and Wang, P., “Lenvatinib in Management of Solid Tumors,” Oncologist 25:e302-e310 (2020), which is hereby incorporated by reference in its entirety) and the PI3K inhibitor buparlisib (FIGS. 5E-5F), which is in phase II trials for dedifferentiated thyroid cancer. Borson-Chazot et al., “Effect of Buparlisib, a Pan-class i pi3k Inhibitor, in Refractory Follicular and Poorly Differentiated Thyroid Cancer,” Thyroid 28:1174-1179 (2018), which is hereby incorporated by reference in its entirety. While MAPK signaling does not directly affect glycogen metabolism, both buparlisib and lenvatinib directly target upstream regulators of GYS1, resulting in slight drug profile overlap with CP. Furthermore, CP did not achieve synergy with the cell cycle inhibitor palbociclib or the topoisomerase inhibitor doxorubicin (FIGS. 5G-5J). Synergy studies were also performed with other metabolic inhibitors to further elucidate the mechanism of cell death following CP treatment. Combination of CP with 2-DG resulted in CDI values close to 1.0 (FIGS. 6K-6L), which suggests drug additivity and redundant inhibition, expected from limiting G6P production from both agents. An alternative hexokinase inhibitor, 3-bromopyruvic acid (3-BP), also failed to achieve synergy with CP (FIGS. 5M-5N). The PPP were then inhibited directly using the G6PDH inhibitor, 6-aminonicotinamide (6-AN). Unlike glycolysis inhibitors, CP and 6-AN achieved remarkable drug synergy (FIGS. 5O-5P), potentially by inhibiting G6PDH activity and substrate availability in concert. Substituting CP for 6-AN support these findings; 6-AN and 2-DG displayed drug antagonism while 6-AN and sorafenib exhibited drug synergy (FIGS. 13A-13B).

### Example 10-CP-91,149 Induces Apoptosis In Vivo to Restrict ATC Tumor Growth

In order to validate the cell-based findings in vivo, nude mice were injected subcutaneously with 8505C in each flank to monitor tumor growth. Once palpable tumors formed (~100 mm³, 10 days post injection), mice were given intraperitoneal injections of vehicle, CP, sorafenib, or combination of CP and sorafenib 6-7 days a week. Tumor growth was notably stunted at approximately equal levels in the CP, sorafenib, and combination group (FIGS. 6A-6D). Although the potential for drug synergy was masked due to the equivalent efficacy with sorafenib, the results demonstrate that CP was equally as effective as sorafenib. Importantly, CP displayed low toxicity, as all mice steadily gained weight over the course of treatment, demonstrating the potential for clinical translation (FIG. 6E). Following completion of the xenograft, tumors were excised and prepared for immunofluorescence experiments for proliferation and apoptosis. Xenografts from mice treated with CP alone or in combination with sorafenib showed a reduction in Ki-67 expression (FIGS. 6F-6G), and CP profoundly induced apoptosis as evident by the amount of PARP cleavage (FIGS. 6F and 6H). In agreement with the in vitro data, CP significantly increased the level of glycogen in the tumor tissue (FIGS. 6F and 6I). Surprisingly, the combination treatment significantly decreased KI-67 expression but did not demonstrate a significant increase in PARP cleavage or glycogen staining (FIGS. 6F-6I). This could be reflective of late-stage apoptosis or even necrosis where cleaved PARP and glycogen are fully degraded by lysosomal hydrolases. Salvesen, G. S., “A Lysosomal Protease Enters the Death Scene, The Journal of Clinical Investigation 107:21-23 (2001), which is hereby incorporated by reference in its entirety. While further studies will be required to investigate specifically how environmental factors such as diet and exercise could impact a glycogen-targeted therapeutic regimen, these data underscore the strong potential for cancer strategies targeting glycogen phosphorylase.

### Example 11—Discussion of Examples 1-10

Successful tumor progression relies on metabolic reprogramming for enhanced energy reserves and a larger supply of metabolic building blocks for daughter cells. Nearly all cancer cells exhibit the Warburg effect to meet these high metabolic needs. Potter et al., “The Warburg Effect: 80 Years on,” Biochemical Society Transactions 44:1499-1505 (2016), which is hereby incorporated by reference in its entirety. Unfortunately, directly inhibiting glycolysis with small molecule inhibitors such as 2-DG or 3-BP have resulted in unacceptable side effects. Ko et al., “A Translational Study “Case Report” on the Small Molecule “Energy Blocker” 3-Bromopyruvate (3 bp) as a Potent Anticancer Agent: From Bench Side to Bedside,” J Bioenerg Biomembr 44:163-170 (2012) and Laussel, C., and Léon, S., “Cellular Toxicity of the Metabolic Inhibitor 2-deoxyglucose and Associated Resistance Mechanisms,” Biochem Pharmacol 182:114213 (2020), both of which are hereby incorporated by reference in their entirety. Therefore, it may be more advantageous to target an altered metabolic pathway in cancer cells that is not active in every cell, such as glycogen metabolism.

Glycogen metabolism is emerging as an important process in cancer biology, as it has been found to play oncogenic roles in diverse tumor types. There are conflicting reports in the literature on the role and quantity of tumor glycogen compared to healthy, normal tissue counterparts. For example, liver cells, which rely on glycogen for gluconeogenesis to supply the body with glucose, have significantly less glycogen than liver cancer cells. Lea et al., “Glycogen Metabolism in Regenerating Liver and Liver Neoplasms,” Cancer Research 32:61-66 (1972), which is hereby incorporated by reference in its entirety. On the other hand, cancer cells from tissues normally devoid of glycogen appear to upregulate glycogen stores. Rousset et al., “Presence of Glycogen and Growth-related Variations in 58 Cultured Human Tumor Cell Lines of Various Tissue Origins,” Cancer Research 41:1165-1170 (1981), which is hereby incorporated by reference in its entirety. Perhaps the role and abundance of glycogen in specific cancer types are linked to the level of dedifferentiation; since healthy liver cells contain the most glycogen of any human cell, liver cancer cells subvert glycogen functionality to instead supply only the cancer cell with glucose. Conversely, normal breast cells are low in glycogen while breast cancer cells may begin to synthesize glycogen for times of low nutrient viability and hypoxia. Favaro et al., “Glucose Utilization Via Glycogen Phosphorylase Sustains Proliferation and Prevents Premature Senescence in Cancer Cells,” Cell Metabolism 16:751-764 (2012) and Markopoulos et al., “Glycogen-rich Clear Cell Carcinoma of the Breast,” World Journal of Surgical Oncology 6:44 (2008), both of which are hereby incorporated by reference in their entirety. It was found that normal thyroid tissues are rich in glycogen (FIG. 1G), and glycogen stores decrease with each step of dedifferentiation towards ATC progression (FIG. 1H). This raises the provocative implication that glycogen may have a physiological role in the thyroid and an oncogenic role in thyroid cancer. The cytoplasm of normal thyrocytes is one of the most oxidative environments in the body due to the production of H₂O₂ to oxidize dietary iodide. Szanto et al., “H₂O₂ Metabolism in Normal Thyroid Cells and in Thyroid Tumorigenesis: Focus on Nadph Oxidases,” Antioxidants (Basel) 8 (2019), which is hereby incorporated by reference in its entirety. In agreement with observations that inhibition of glycogenolysis results in ROS accumulation in ATC cells (FIG. 4L), the role of glycogen in normal thyroid cells could be to protect against ROS formed during thyroid hormone synthesis. Further studies will be required to determine the exact fate of glycogen-derived carbon in thyroid cancer compared to normally functioning thyrocytes.

Although glycogen anabolism and catabolism represent exciting drug targets in cancer biology, few studies have investigated this potential. CP-91,149 was originally developed to treat diabetes by binding to the indole-site of glycogen phosphorylase, blocking the homodimer interface. Martin et al., “Discovery of a Human Liver Glycogen Phosphorylase Inhibitor That Lowers Blood Glucose In vivo,” Proc Natl Acad Sci USA 95:1776-1781 (1998), which is hereby incorporated by reference in its entirety. CP-91,149 inhibits all PYG isoforms and crosses the blood brain barrier for potential application in treating brain cancers. However, PYG inhibitors could be better optimized to suit the metabolic profile of different tumors. For example, potential toxicity could be avoided by exclusively targeting PYGB over PYGL. Nevertheless, no side effects have been reported following CP-91,149 treatment in mice; 50-100 mg/kg of CP-91,149 decreased obese mouse blood glucose levels but not those of lean mice, and it is reported here that all mice steadily gained weight regardless of treatment (FIG. 6E).

Inhibiting glycogen phosphorylase has garnered exciting in vitro results in diverse cancer cell lines. Pancreatic adenocarcinoma cells were found to be sensitive to CP-320626, a PYG inhibitor similar in structure to CP-91,149. Lee et al., “Metabolic Sensitivity of Pancreatic Tumour Cell Apoptosis to Glycogen Phosphorylase Inhibitor Treatment,” British Journal of Cancer 91:2094-2100 (2004), which is hereby incorporated by reference in its entirety. CP-320626 inhibited cellular respiration, the PPP, anaplerosis, and synthesis of ribose and fatty acids, indicating a major redistribution of carbon due to glycogenolysis inhibition. Studies in cell lines from glioblastoma, breast cancer, and colon cancer revealed that GYS1 and PYGL expression were increased in response to hypoxia. Favaro et al., “Glucose Utilization Via Glycogen Phosphorylase Sustains Proliferation and Prevents Premature Senescence in Cancer Cells,” Cell Metabolism 16:751-764 (2012), which is hereby incorporated by reference in its entirety. Furthermore, knocking down PYGL induced ROS accumulation and senescence in cancer cells. In hepatocellular carcinoma, CP-91,149 also increased ROS levels but triggered autophagic adaptations. Barot et al., “Inhibition of Glycogen Catabolism Induces Intrinsic Apoptosis and Augments Multikinase Inhibitors in Hepatocellular Carcinoma Cells,” Experimental Cell Research 381(2):288-300 (2019), which is hereby incorporated by reference in its entirety. In agreement with the findings (FIGS. 5A-B and 5K-5N), CP-91,149 treatment in HepG2 cells displayed synergy with sorafenib but not 2-DG or 3-BP. Fewer studies have been conducted on inhibiting glycogen synthase in cancer, as most pharmacological strategies target the upstream regulator of GYS1, glycogen synthase kinase 3. Guaiacol, a naturally occurring flavoring agent, was recently identified as a direct inhibitor of GYS1 by competing with the substrate UDP-glucose for incorporation into the growing glycogen chain. Kakhlon et al., “Guaiacol as a Drug Candidate for Treating Adult Polyglucosan Body Disease,” JCI Insight 3: e99694 (2018), which is hereby incorporated by reference in its entirety. Guaiacol decreased glycogen stores in mouse embryonic fibroblasts by 50%, which is comparable to guaiacol treatment in ATC cells (FIG. 10G). A recent synthetic GYS1 inhibitor, yGsy2p-IN-1, robustly inhibited purified GYS1 and decreased 14C-glucose incorporation in cell lysates. Tang et al., “Discovery and Development of Small-molecule Inhibitors of Glycogen Synthase,” J Med Chem 63:3538-3551 (2020), which is hereby incorporated by reference in its entirety. It is reported here that yGsy2p-IN-1 successfully reduced glycogen levels and limited cell viability in ATC cells (FIGS. 10H-10I), demonstrating the inhibitor's antimetabolic effects in vitro. Further studies are needed to better elucidate optimal drug combinations with a glycogen metabolism modulator based on the genetic background and cell signaling landscape of individual tumors.

Although ATC is one of the most lethal solid tumors with no long-term therapeutic option, no study thus far has directly investigated glycogen in normal thyroid or thyroid cancer cells. It is reported that normal thyroid tissue as well as thyroid adenoma, PTC, FTC, and ATC express the genes and enzymes necessary to metabolize glycogen (FIGS. 1A-1H and 2A-2I). Interestingly, normal thyroid tissue expressed PYGL but not PYGB. While PYGL and PYGB exhibit comparable enzyme kinetics, they have different mechanisms of allosteric regulation. PYGL is more dependent on hormone signaling from insulin and glucagon, while PYGB activity is more impacted through intracellular nutrient availability, e.g., G6P and AMP/ATP ratio. Mathieu et al., “The Structure of Brain Glycogen Phosphorylase—from Allosteric Regulation Mechanisms to Clinical Perspectives,” The FEBS journal 284:546-554 (2017), which is hereby incorporated by reference in its entirety. PYGB may confer an oncogenic advantage over PYGL; instead of responding to hormones to selflessly catabolize glycogen for the body, PYGB is expressed to directly serve the metabolic needs of the cancer cell. Since PYGB overexpression has been observed in several aggressive tumors such as colorectal carcinoma, non-small lung cancer, metastatic breast cancers, and now anaplastic thyroid cancer, PYGB expression and the concomitant shunt in glycogen catabolism may represent a crucial step in successful tumor progression. Altemus et al., “Breast Cancers Utilize Hypoxic Glycogen Stores Via pygb, the Brain Isoform of Glycogen Phosphorylase, to Promote Metastatic Phenotypes,” PLoS One 14:e0220973 (2019); Lee et al., “Clinicopathological Significance of bgp Expression in Non-small-cell Lung Carcinoma: Relationship With Histological Type, Microvessel Density and Patients' Survival,” Pathology 38:555-560 (2006); and Tashima et al., “Expression of Brain-type Glycogen Phosphorylase is a Potentially Novel Early Biomarker in the Carcinogenesis of Human Colorectal Carcinomas,” Am J Gastroenterol 95:255-263 (2000), all of which are hereby incorporated by reference in their entirety. The levels of glycogen in the thyroid tissue samples were depleted with PYGB expression, which can be inhibited with small molecule inhibitors to cause glycogen buildup (FIGS. 3A-3E). A lack of available glycogen causes a depletion of NADPH and GSH, a rise in ROS levels, impaired OCR, and apoptosis induction (FIGS. 3G, 4J-4K, and 4N-4O). Based on these results, it is proposed that glycogen-derived carbon preferentially fuels the PPP over glycolysis (FIG. 7). Further studies are needed to investigate the exact mechanism of directing this differential carbon trafficking. One explanation may be that the colocalization of glycogen granules and enzymes known as the glycosome is localized to the endoplasmic reticulum. Glycogen has been shown to be associated with ER membranes, and cancer cells rely heavily on the PPP in the ER to combat the unfolded protein response. Jerome, W. G., and Cardell, R. R., “Observations on the Role of Smooth Endoplasmic Reticulumin Glucocorticoid-induced Hepatic Glycogen Deposition,” Tissue Cell 15:711-727 (1983); Lytridou et al., “Stbd1 Promotes Glycogen Clustering During Endoplasmic Reticulum Stress and Supports Survival of Mouse Myoblasts,” J Cell Sci 133(20):jcs244855 (2020); and Mandl, J., and Bánhegyi, G., “The er-Glycogen Particle-Phagophore Triangle: A Hub Connecting Glycogenolysis and Glycophagy?,” Pathology & Oncology Research 24:821-826 (2018), all of which are hereby incorporated by reference in their entirety. Excitingly, CP-91,149 achieved profound drug synergy with sorafenib in vitro and significantly impaired tumor growth in vivo (FIGS. 5A-5B and 6A-6E). Taken together, these results expose glycogen metabolism to be a novel metabolic vulnerability in ATC cells.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating a solid tumor cancer in a subject, said method comprising: administering to the subject an inhibitor of glycogen metabolism under conditions effective to treat the cancer, wherein the solid tumor cancer is characterized by cells having an increased glycogen expression or activity relative to corresponding non-cancer cells of similar origin.

2. (canceled)

3. (canceled)

4. The method of claim 1, wherein the subject does not have or is not suspected of having Diabetes mellitus.

5. (canceled)

6. The method of claim 1, wherein the cancer is selected from the group consisting of thyroid cancer, breast cancer, colorectal cancer, bladder cancer, cervical cancer, esophageal cancer, gastric cancer, head and neck cancer, kidney cancer, liver cancer, lung cancer, nasopharyngeal cancer, ovarian cancer, cholangiocarcinoma, pancreatic cancer, prostate cancer, glioblastoma, astrocytoma, melanoma, mesothelioma, musculoskeletal sarcoma, and soft tissue sarcoma.

7. The method of claim 1, wherein the cancer is thyroid cancer.

8. The method of claim 7, wherein the thyroid cancer is advanced anaplastic thyroid cancer, poorly differentiated thyroid cancer, metastatic thyroid cancer, treatment resistant thyroid cancer, or recurrent thyroid cancer.

9. The method of claim 1, wherein the cancer is breast cancer.

10. The method of claim 9, wherein the breast cancer is triple negative breast cancer, estrogen receptor-positive breast cancer, metastatic breast cancer, HER2 positive breast cancer, or variants thereof.

11. (canceled)

12. (canceled)

13. The method of claim 1, wherein the inhibitor of glycogen metabolism is an inhibitor of glycogen phosphorylase.

14. The method of claim 13, wherein the inhibitor of glycogen phosphorylase is selected from 5-chloro-N-[(2S,3R)-4-(dimethylamino)-3-hydroxy-4-oxo-1-phenylbutan-2-yl]1H-indole-2-carboxamide (CP-91149), 5-chloro-N-[(2S,3R)-4-[(3R,4S)-3,4dihydroxypyrrolidin-1-yl]-3-hydroxy-4-oxo-1-phenylbutan-2-yl]-1H-indole-2-carboxamide (ingliforib), 5-chloro-N[(2S)-3-(4-fluorophenyl)-1-(4-hydroxypiperidin-1-yl)-1-oxopropan-2-yl]1H-indole-2-carboxamide (CP-320626), (2R,3S)-2,3-bis[[(E)-3-(4-hydroxyphenyl) prop-2-enoyl]oxy]pentanedioic acid (FR258900), N-(3,5-dimethyl-benzoyl)-N′-([β-D-glucopyranosyl) urea (KB228), 5-chloro-N-[(2S,3R)-3-hydroxy-4-[methoxy(methyl)amino]-4-oxo-1-phenylbutan-2-yl]-1H-indole-2-carboxamide (CP-316819), 5-chloro-N-[3-(4-fluorophenyl)-1-(4-hydroxypiperidin-1-yl)-1-oxopropan-2-yl]-1H-indole-2-carboxamide (CP320626), isopropyl 4-(2-chlorophenyl)-1-ethyl-2-methyl-5-oxo-1,4,5,7-tetrahydro-furo[3,4-b]pyridine-3-carboxylate (BAY R3401), (4S)-1-ethyl-6-methyl-4-phenyl-5-propan-2-yloxycarbonyl-4H-pyridine-2,3dicarboxylic acid (BAY-W1807), 1,4-dideoxy-1,4-amino-D-arabinitol (DAB), 4-[3-(2-Chloro-4,5-Difluoro-Benzoyl) ureido]-3-Trifluoromethoxybenzoic Acid (AVE5688), GSK1362885, PSN-357, or any combination thereof.

15. The method of claim 13, wherein the inhibitor of glycogen phosphorylase is selected from 5-chloro-N-[(2S,3R)-4-(dimethylamino)-3-hydroxy-4-oxo-1-phenylbutan-2-yl]1H-indole-2-carboxamide (CP-91149).

16. The method of claim 1 further comprising:
administering an additional cancer therapy.

17. The method of claim 16, wherein the additional cancer therapy is selected from a MAPK inhibitor, a pentose phosphate pathway inhibitor, an inhibitor of cancer stem cell formation, a cyclin dependent kinase (CDK) inhibitor, a tyrosine kinase inhibitor, a thyroid hormone receptor beta-1 (TRβ) agonist, an anthracycline antibiotic, a glucose molecule, a pyruvic acid derivative, a phosphogluconate dehydrogenase inhibitor, an activator of Interferon/JAK1/STAT1 signaling, a phosphoinositide 3-kinase (PBK) inhibitor, a PTEN activator, an anti-estrogen, or a combination thereof.

18.-31. (canceled)

32. The method of claim 16, wherein the additional cancer therapy is selected from Sorafenib, Lenvatinib, Bevacizumab, Ranibizumab, Aflibercept, Sunitinib, Pazopanib, Axitinib, Regorafenib, Nintedanib, or a combination thereof.

33. The method of claim 1, wherein the amount of the inhibitor of glycogen metabolism administered is between about 1.0 mg/kg and about 100 mg/kg.

34. A method of inhibiting tumor growth in a subject, said method comprising:
administering to a subject having a tumor an inhibitor of glycogen metabolism under conditions effective to treat the tumor, wherein the tumor is characterized by cells having an increased glycogen expression or activity relative to corresponding non-tumor cells of similar origin.

35.-38. (canceled)

39. The method of claim 34, wherein the tumor comprises a cancer selected from the group consisting of thyroid cancer, breast cancer, colorectal cancer, bladder cancer, cervical cancer, esophageal cancer, gastric cancer, head and neck cancer, kidney cancer, liver cancer, lung cancer, nasopharyngeal cancer, ovarian cancer, cholangiocarcinoma, pancreatic cancer, prostate cancer, glioblastoma, astrocytoma, melanoma, mesothelioma, musculoskeletal sarcoma, and soft tissue sarcoma.

40. The method of claim 34, wherein the tumor comprises thyroid cancer.

41. The method of claim 40, wherein the thyroid cancer is advanced anaplastic thyroid cancer, poorly differentiated thyroid cancer, metastatic thyroid cancer, treatment resistant thyroid cancer, or recurrent thyroid cancer.

42.-84. (canceled)

85. The method of claim 7, wherein the inhibitor of glycogen metabolism is 5-chloro-N-[(2S,3R)-4-(dimethylamino)-3-hydroxy-4-oxo-1-phenylbutan-2-yl]1H-indole-2-carboxamide (CP-91149) and the subject is further administered sorafenib.
